(19) United States
(12) Patent Application Publication
(10) Pub. No.: US 2023/0416602 A1
(43) Pub. Date: Dec. 28, 2023

(54) FLUORESCENT POLYMERS AND SOLUTIONS THEREOF FOR SCALE CONTROL IN AQUEOUS SYSTEMS

(71) Applicant: Nouryon Chemicals International B.V., Arnhem (NL)

(72) Inventor: Klin Aloysius RODRIGUES, Arnhem (NL)

(73) Assignee: Nouryon Chemicals International B.V., Arnhem (NL)

(21) Appl. No.: 18/038,089

(22) PCT Filed: Nov. 22, 2021
(86) PCT No.: PCT/IB2021/000806

§ 371 Date: May 22, 2023

Related U.S. Application Data
(60) Provisional application No. 63/228,223, filed on Aug. 2, 2021.
(60) Provisional application No. 63/116,428, filed on Nov. 20, 2020.

(30) Foreign Application Priority Data
Jan. 27, 2021 (EP) 21153726.1

(51) Int. Cl. C09K 11/06; C07D 311/82; C08F 220/58; C09K 15/28; C11D 3/37; C11D 3/40; C07D 311/16; B08B 3/08; C02F 5/10; C02F 1/56

(52) U.S. Cl. CPC C09K 11/06; C07D 311/82; C08F 220/585; C09K 15/28; C11D 3/378; C11D 3/40; C07D 311/16; B08B 3/08; C02F 5/10; C02F 1/56; C09K 2211/145; C09K 2211/1425; C02F 2303/08; C02F 2103/023

(57) ABSTRACT

Disclosed are fluorescent water-soluble water treatment polymers suitable for use in scale inhibition or suppression of corrosion in industrial water systems, the water treatment polymers especially comprising fluorescent coumarin, fluorescein, rhodamine, and Nile blue derivative monomers. Also disclosed are methods of making the monomers, methods of making the polymers, methods of inhibiting scale in an industrial water system, methods of suppressing corrosion in an industrial water system, and methods of using the polymers in coagulation and flocculation, and in cleaning applications.

### PRIORITY CLAIM

This application claims priority of U.S. Provisional Application Ser. No. 63/228,223, filed Aug. 2, 2021, European Application No. 21153726.1, filed Jan. 27, 2021, and U.S. Provisional Application Ser. No. 63/116,428, filed Nov. 20, 2020, the entire contents of which applications are hereby incorporated herein by reference.

### FIELD OF THE DISCLOSURE

This application relates to methods of making water-soluble fluorescent water treatment polymers comprising fluorescent monomers, to the fluorescent water treatment polymers obtainable by such methods, and aqueous solutions containing them, and their application in a method for controlling scale or suppressing corrosion in industrial water systems by treatment with the water-soluble fluorescent water treatment polymer containing the fluorescent monomer, and their use as an additive for coagulation or flocculation, or in cleaning applications. This application further relates to fluorescent monomers that are suitable as starting compounds or intermediates in the method of making water-soluble fluorescent water treatment polymers, and to compositions comprising such monomers that are suitable as premixes to be employed in the method of making water-soluble fluorescent water treatment polymers.

### BACKGROUND

There are many industrial water systems, including, but not limited to, cooling water systems and boiler water systems. Such industrial water systems are subject to corrosion and the formation of scale.

It is known that certain types of water-soluble treatment polymers are effective for inhibiting scale and suppressing the occurrence of corrosion in industrial water systems. These water-soluble treatment polymers are known to persons of ordinary skill in the art of industrial water systems and are widely used in scale inhibition products. Such water-soluble treatment polymers generally exhibit activity against scale when added to water in an amount in the range of from about 1 to about 100 ppm.

The efficacy of water-soluble treatment polymers in inhibiting scale and suppressing corrosion depends in part on the concentration of the water-soluble treatment polymer in the water system. Water-soluble treatment polymers added to an industrial water system can be consumed by many causes, leading to changes in concentration of the water-soluble treatment polymer. Therefore, it is important for the optimum operation of an industrial water system to be able to accurately determine the concentration of water-soluble treatment polymers in the water.

It is known that the concentration of water-soluble treatment polymers used as components of scale and corrosion inhibitors in industrial water systems can be monitored if the polymer is tagged with a fluorescent monomer. The amount of fluorescent monomer incorporated into the water-soluble polymer must be enough so that the fluorescence of the water-soluble polymer can be adequately measured, however, it must not be so much as to adversely impact the performance of the water-soluble polymer as a treatment agent. Because the concentration of the tagged water-soluble treatment polymer can be determined using a fluorimeter, it is also possible to measure consumption of the water-soluble treatment polymer directly. It is important to be able to measure consumption directly because consumption of a water-soluble treatment polymer usually indicates that a non-desired event, such as scaling, is occurring. Thus, by being able to measure consumption of the water-soluble treatment polymer, there can be achieved an in-line, real time in situ measurement of scaling activity in the industrial water system. Such in-line, real time measurement systems are disclosed, for example, in U.S. Pat. Nos. 5,171,450, 5,986,030, and 6,280,635, all of which are incorporated herein by reference.

A wide array of water treatment formulations will also contain phosphate to minimize corrosion. Many states now have regulations limiting the amount of phosphates that can be used in water treatment systems or otherwise be potentially released to the environment. Even in states where the use of phosphate is allowed, it is considered desirable to minimize the amount of phosphate released to the environment. Therefore, the use of higher pH water systems that are lower in phosphates is becoming more common. But such higher pH water systems lead to increased carbonate scaling. Therefore, there is a need for methods of controlling carbonate scale in industrial water systems, particularly in higher pH environments.

It is further known in the art that some water-soluble treatment polymers will be more effective in the inhibition of phosphate scale, while other water-soluble treatment polymers will be more effective in the inhibition of carbonate scale. Yet others will be more effective in the inhibition of silica and silicates scales, and still others will be effective in the inhibition of sulfate scale.

Naphthalimide and certain naphthalimide derivatives are known fluorescent compounds that can be converted to polymerizable fluorescent monomers for use in such systems.

Because water-soluble treatment polymers are typically polymerized in an aqueous medium, it has been known to use water-soluble naphthalimide derivative monomers in the manufacture of such water treatment polymers, as shown, for example, in U.S. Pat. No. 6,645,428, which discloses water-soluble quaternized naphthalimide derivative monomers and the use thereof to prevent or reduce phosphate scale. The process described in US '428 has as an additional major disadvantage that we've discovered—the monomers are not completely reacted into the polymer and stay in the product. As the monomers also contain the fluorescent naphthalimide unit, this makes the fluorescent signal unreliable when used in water treatment.

It thus would be desirable to provide compositions and methods for controlling scale, mainly carbonate scale and phosphate scale, in industrial water systems comprising treating with a water-soluble fluorescent water treatment polymer containing a fluorescent monomer which polymer provides a reliable detectable fluorescent signal under typical industrial water treatment conditions, and methods of making such polymers.

It further would be desirable to provide fluorescent monomers based on other chemistries, to provide a process to employ them and convert them into such water-soluble water treatment polymers, and methods of making such monomers.

### SUMMARY

In one aspect, the present disclosure relates to a fluorescent monomer of Structure I which is substantially free of Structure II, wherein Structure I comprises rings A and B and optionally rings C and/or D:

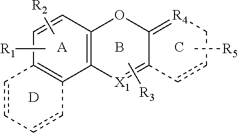

wherein
R₁ is H, alkyl, aryl, arylalkyl, aryloxy, NH₂, NHalkyl, N(alkyl)₂, =NH⁺alkyl X⁻, ═N(alkyl)₂X⁻, =NH₂+X⁻, (meth)acryloyloxy, vinylaryl, vinylarylalkyl, vinylaryloxy, vinylarylalkyloxy, (meth)allyl, (meth)allyloxy, (meth)acryloxy, (meth)acryloxyalkyl, (meth)acryloxypolyalkylene oxide, (meth)allylamino, (meth)acrylamido, polyalkylene oxide, oxo, OH, O⁻M⁺, alkoxy, CₙH₂ₙ₊₁CH═CH— alkylene-, CₙH₂ₙ₊₁CH═C(CH₃)-alkylene-, CₙH₂ₙ₊₁CH═CH-alkylene-O—, CₙH₂ₙ₊₁CH═C(CH₃)-alkylene-O—, —COOH, -alkylene-COOH, —COO⁻M⁺, -alkylene-COO⁻M⁺, or —O-alkylene-(meth)acrylate;
R₂ is H or alkyl;
R₃ is H, alkyl, aryl, arylalkyl, aryloxy, (meth)allyl, (meth)allylamino, (meth)allyloxy, (meth)allyloxyalkoxide, (meth)acryloyloxy, ((meth)acryloxyalkyl, meth)acryloxypolyalkylene oxide, (meth)acryloyloxyalkoxide, polyalkylene oxide, vinylaryl, vinylarylalkyl, vinylaryloxy, vinylarylalkyloxy, —OH, or —O⁻M⁺, alkoxy, —COOH, -alkylene-COOH, —COO⁻M⁺, —CH₂COO⁻M⁺, CH₃(CH₂)ₙ₂C(O)OCₙH₂ₙ₊₁CH═CHCH₂—, CH₃(CH₂)ₙ₂C(O)O or CₙH₂ₙ₊₁CH═C(CH₃)CH₂—;
or R₃ is

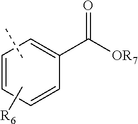

where the dotted bond joins R₃ to the remainder of Structure I;
X₁ represents CH or N;
R₄ is O when ring C is absent; or represents CH when ring C is present;
R₅ is =NH₂+X⁻, =NH⁺alkyl X⁻, ═N⁺(alkyl)₂X⁻, NH₂, NHalkyl, N(alkyl)₂, or ═O;
R₆ is H, alkyl, alkylene, —NH₂, NHalkyl, N(alkyl)₂, (meth)acryloxy, (meth)acrylamido, (meth)allyl, —COOH, —COO⁻M⁺, phosphate, phosphonate, sulfonate, vinylaryl, vinylarylalkyl, vinylaryloxy, vinylarylalkyloxy, (meth)allyloxy, (meth)acryloxy, (meth)acryloxypolyalkylene oxide, (meth)acrylamido, polyalkylene oxide, (meth)acryloxyalkyl, or (meth)allylamino;
R₇ is H, M⁺, alkyl, alkenyl, arylalkyl, alkyl-CH═CH-alkylene-, alkyl-CH═C(CH₃)-alkylene-, —O-(meth)-alkyl, CH₂═CR′—C(═O)—O-alkylene-O—C(═O)—O—, (meth)allyloxy, (meth)acryloxy, (meth)acryloxypolyalkylene oxide, (meth)acrylamido, polyalkylene oxide, (meth)acryloxyalkyl, or (meth)allylamino;
R′ is H or alkyl;
n is 0-10;
m is 2 when n=0; and m is 1 when n=1-10;
X⁻ is an anionic counter ion; and
M⁺ is a cationic counterion;
with the proviso that R₁, R₃, R₆ or R₇ have at least one polymerizable double bond;
and Structure II comprises rings E and F and optionally rings G and/or H:

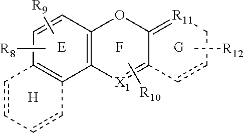

wherein
R₈ is H, alkyl, NH₂, NHalkyl, N(alkyl)₂, =NH₂+X⁻, =NH⁺alkyl X⁻, ═N(alkyl)₂X⁻, ═O, OH, or O⁻M⁺, —COOH, -alkylene-COOH, or —COO⁻M⁺, or -alkylene-COO⁻M⁺;
R₉ is H or alkyl;
R₁₀ is H, —OH, or —O⁻M⁺, alkyl, —COOH, -alkylene-COOH, —COO⁻M⁺, or -alkylene-COO⁻M⁺;
or R₁₀ is

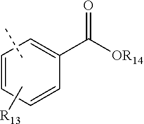

where the dotted bond joins R₁₀ to the remainder of Structure II;
X₁ is CH or N;
R₁₁ is O when ring G is absent; or represents CH when ring G is present;
R₁₂ is =NH₂+X⁻, =NH⁺alkyl X⁻, ═N₊(alkyl)₂X⁻, NH₂, NHalkyl, N(alkyl)₂, or ═O;
R₁₃ is H, —NH₂, NHalkyl, N(alkyl)₂, —COOH, —COO⁻M⁺, phosphate, phosphonate, or sulfonate;
R₁₄ is H or M⁺;
X⁻ has the meaning given above;
M⁺ has the meaning given above; and
   rings D and H, when present, are optionally substituted.

Throughout this disclosure, X⁻ is an anionic counter ion, and is preferably selected from but not limited to chloride, bromide, hydroxide, methosulphate, sulfate, sulfonate, carboxylate, chlorate, phosphate, and phosphonate.

Throughout this disclosure, M⁺ is a cationic counterion, and is preferably selected from but not limited to sodium, potassium, ammonium, and amine salt.

Throughout this disclosure, “polyalkylene oxide,” whether alone or as a part of any group, unless otherwise indicated, means H-(alkylene-O)ᵣ— where “r” is the number of repeating units, typically 1-20 repeat units, preferably 1-10 repeat units, more preferably 1-3 repeat units. Where the disclosure refers to “polyethylene oxide,” whether alone or as a part of any group, it is to be understood in a similar manner, unless otherwise indicated, to mean H—(CH₂CH₂—O)ᵣ— where “r” is the number of repeating units, typically 1-20 repeat units, preferably 1-10 repeat units, more preferably 1-3 repeat units.

In one aspect, this disclosure relates to methods of making water-soluble fluorescent water treatment polymers wherein fluorescent coumarin, fluorescein, rhodamine, and Nile blue derivative monomers are polymerized. In another embodiment of the method, the polymerization reaction takes place in an aqueous reaction medium. In another embodiment of the method, the polymerization reaction takes place in a non-aqueous reaction medium. In yet another aspect, this disclosure relates to aqueous compositions comprising water-soluble fluorescent polymers obtainable by the above method, suitable for use as a water treatment polymer, wherein the polymer comprises fluorescent coumarin, fluorescein, rhodamine, or Nile blue derivative monomer. The water-soluble polymer can be present in the aqueous composition as at least 10 wt %.

In one aspect this disclosure relates to a method of treating an industrial water system to aid in inhibiting the deposition of scale, the method comprising treatment of the industrial water system with a water-soluble fluorescent water treatment polymer wherein the polymer comprises fluorescent coumarin, fluorescein, rhodamine, or Nile blue derivative monomer.

In one aspect, this disclosure relates to compositions comprising selected fluorescent coumarin, fluorescein, rhodamine, or Nile blue derivative monomer that are suitable premixes for performing the above method of making water-soluble fluorescent water treatment polymers.

In one aspect, the disclosure relates to an aqueous composition comprising a water-soluble fluorescent water treatment polymer wherein the polymer comprises at least one carboxylic acid monomer and fluorescent coumarin, fluorescein, rhodamine, or Nile blue derivative monomer.

In one aspect this disclosure relates to a method of controlling scale in a water system.

In one aspect this disclosure relates to a method of suppressing corrosion in a water system.

In one aspect this disclosure relates to a method of coagulation or flocculation in a water treatment system.

In one aspect this disclosure relates to a method of cleaning.

It should be noted that fluorescein below can exist in equilibrium between two forms: an “open” form and a “closed” form.

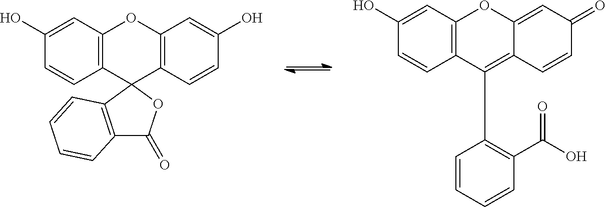 [SMILES: O=C1OC2(c3ccc(O)cc3Oc3cc(O)ccc32)c2ccccc21 ; O=C(O)c1ccccc1-c1c2ccc(=O)cc-2oc2cc(O)ccc12]

Rhodamine B below can exist in equilibrium between two forms: an “open” form and a “closed” form. The “open” form dominates in acidic condition while the “closed” form is in basic conditions.

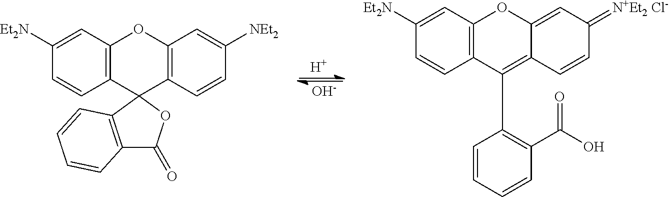

For purposes of this disclosure, we will illustrate the open form which typically is the more fluorescent moiety. Nevertheless, it is understood that at any given condition (pH, temperature etc.) an equilibrium exists and both forms may be present. Therefore, the closed form will also be covered by this disclosure.

Similarly, other fluorescent monomers, for example, Nile blue, may adopt different equilibrium structures depending on conditions and one or more equilibrium forms may be present. This disclosure covers all such equilibrium forms.

For example, Nile blue can exist between these two equilibrium structures in the specification:

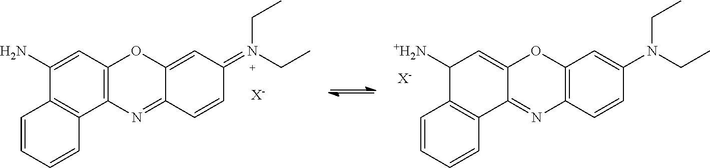

It is intended that both equilibrium structures are covered wherever the claims cover one such equilibrium structure.

The other monomers of the fluorescent water treatment polymers as disclosed herein can be selected to provide water treatment polymers that are effective in the inhibition of any one or more of carbonate scale, phosphate scale, silica scale, and sulfate scale, most importantly carbonate scale and phosphate scale.

### DETAILED DESCRIPTION

As used herein, the term “dosing” of a reactant into a reaction mixture means that the reactant is added over a period of time during the course of the reaction, as opposed to a single addition of an entire reactant portion. As used herein, the term “dosing” of a reactant into a reaction mixture encompasses addition of a reactant to a reaction mixture as a continuous stream, addition of a reactant into a reaction mixture as several intermittent shots, and combinations thereof.

As used herein, the term “water-soluble” with respect to the fluorescent water treatment polymers disclosed herein means that the fluorescent water treatment polymers have a water solubility of at least 10 grams per 100 mls of water at 25° C., preferably at least 20 grams per 100 mls of water at 25° C., and most preferably at least 30 grams per 100 mls of water at 25° C., all at pH 7.

The water-soluble treatment polymer needs to be pumpable. In a preferred embodiment, the viscosity of the water-soluble treatment polymer needs to be less than 25,000 cps, less than 10,000 cps and preferably less than 5000 cps and most preferably less than 2500 cps at preferably 10, more preferably 20, more preferably 30, most preferably 40% polymer solids at 25° C. at 10 rpm in the pH range 2-10, preferably 3-8 most preferably 4-6.

Substantially free of impurities in Structure (I) means that the impurity of Structure (II) is preferably less than 20%, preferably less than 15%, preferably less than 10%, more preferably less than 5%, and most preferably less than 2% or is undetectable of/in Structure (I) when measured by area percent using a suitable analytical technique such as liquid chromatography.

The mol % determination by LC requires that each compound the synthesized and purified to get a viable LC standard. For purposes of this disclosure, the mol % is correlated to the ranges of area % by LC as shown below.

Unless otherwise indicated, that a first substance is “substantially free” of a second substance, as used herein, means, as discussed above, that the first substance has preferably less than 20 mol % (15-25 area % by LC), preferably less than 15 mol % (10-20 area % by LC), preferably less than 10 mol % (5-15 area % by LC), more preferably less than 5 mol % (2.5-7.5 area % by LC), more preferably less than 3 mol % (1-5 area % by LC), more preferably less than 2 mol %(1-3 area % by LC), and most preferably less than 1.5 mol % (1-2 area % by LC) or is even completely free of the second substance relative to 100 mol % of the first substance.

Unless otherwise indicated, all percentages of a composition, for example, a solid or a solution, are mole percentages based on the total composition.

### Polymerization Methods

There are 3 main processes (Method A, B and C below) that can preferably be used to prepare a water-soluble fluorescent polymer useful in water treatment. Method A is the one that would be most preferred and would be utilized in most cases.

In embodiments of polymerizing the fluorescent water treatment polymers as disclosed herein, it is desirable to maximize the amount of added fluorescent monomer that is polymerized into the polymer. It is preferred that at least 85% of the fluorescent monomer added to the polymerization reaction be converted to the polymer or at least 90%, or at least 92%, or at least 95%, or at least 98%, or at least 99%. It also is desirable to achieve an even distribution of the fluorescent monomer along the polymer backbone. These objectives can be achieved by polymerization methods in which one or more of the monomers or initiators are dosed into the reaction medium at a controlled rate, in accordance with the disclosed embodiments. The choice of polymerization method will depend on the relative solubilities and reactivities of the selected monomers, and the selected solvents.

Method A—Dosing of Fluorescent Monomer, Acid Monomer, and Initiator

One method for polymerization of a water-soluble fluorescent water treatment polymer comprising one or more coumarin, fluorescein, rhodamine, or Nile blue derivative monomers comprises the steps of
a) providing a quantity of fluorescent coumarin, fluorescein, rhodamine, or Nile blue derivative monomer as disclosed herein;
b) dissolving the fluorescent coumarin, fluorescein, rhodamine, or Nile blue derivative monomer in a quantity of a liquid polymerizable carboxylic acid monomer to provide a fluorescent monomer-acid monomer solution;
c) dosing the fluorescent monomer-acid monomer solution into a reaction medium; optionally adding a part of the fluorescent monomer-acid monomer solution to the initial polymerization solution or a part of the fluorescent monomer to the initial polymerization solution;
d) initiating polymerization of dosed monomers in the reaction medium in the presence of a polymerization initiator, and
e) maintaining the dosing of the fluorescent monomer-acid monomer solution into the reaction medium during the polymerization reaction, such that the polymerization reaction continues while the fluorescent monomer-acid monomer solution is being dosed to the reaction medium,
f) optionally adding other acid monomers and/or other monomers after the end of the fluorescent monomer-acid monomer solution has been dosed to maximize the conversion of the fluorescent monomer,
   wherein the polymerization reaction yields a water-soluble fluorescent polymer suitable for use in treatment of an industrial water system.

Optionally, the fluorescent monomer can be dissolved in a solvent that is preferably water miscible or into other non-carboxylic acid monomers and a part of this can be added to the initial polymerization solution and the other part dosed into the polymerization process.

In one embodiment, the reaction medium is aqueous; optionally including co-solvents which can include without limitation dimethyl formamide, methanol, ethanol, isopropanol, n-propanol, glycols, and glycol ethers. In another embodiment, the reaction medium is non-aqueous, with xylene being a preferred non-aqueous reaction medium. The selection of aqueous or non-aqueous reaction medium could depend on the choice of carboxylic acid monomer used. For example, if the carboxylic acid monomer is acrylic acid or methacrylic acid, then an aqueous reaction medium can be preferred, while if the carboxylic acid is maleic acid, itaconic acid, or either of their anhydrides or salts, then a non-aqueous reaction medium can be preferred. Where a non-aqueous reaction medium is used, as a final step the non-aqueous medium is removed, and the reaction product is converted to an aqueous composition. The reaction medium or purification step is preferably free of chlorinated solvents since these are environmentally friendly. The final aqueous solution of the polymer is preferably free of chlorinated solvents. This means that the final aqueous solution of the polymer has less than 1%, less 0.1%, less than 0.01% and most preferably does not have any chlorinated solvents.

This embodiment of the method is useful when the non-fluorescent monomers in the mixture polymerize more rapidly than the fluorescent monomers under the reaction conditions employed. Dosing the more highly reactive monomers into the reaction medium at a controlled rate provides a controlled rate of reaction and more even distribution of the fluorescent monomer along the polymer chain. Otherwise, if the more highly reactive non-fluorescent monomers are fully present at the initiation of the polymerization reaction, then it is possible that the non-fluorescent monomers will react mostly with themselves, with uneven distribution of the fluorescent monomer in the water treatment polymer. It is also possible that relatively large amounts of the fluorescent monomer would remain unpolymerized; such unpolymerized monomers present in a water treatment composition can lead to inaccurate and misleading indications of scale inhibition when such compositions are added to an industrial water system and the resulting fluorescence is measured. Acrylic acid and methacrylic acid both have faster polymerization rates than some of the allylic fluorescent monomers disclosed herein. Therefore, if these monomers are used, then it is preferred to use the method as described above wherein the acid monomer-fluorescent monomer solution is dosed into the reaction medium at a controlled rate.

This method also is advantageous when the fluorescent monomer is a low water-soluble monomer such as coumarin derivatives and the reaction medium is aqueous. The low water-soluble monomer can first be dissolved in the liquid carboxylic acid monomer, and the addition rate of the acid-monomer-fluorescent monomer solution can be controlled so that the fluorescent monomer remains dissolved in the aqueous polymerization reaction medium. This can be observed visually during the reaction, wherein a clear solution indicates that the monomers remain dissolved, and a hazy appearance can indicate that any of the monomers is not dissolved.

One or more additional monomers can be present in the polymerization mixture. The one or more additional monomers can be present in the reaction medium when dosing of the fluorescent monomer-acid monomer solution is begun; or the one or more additional monomers can be present in the fluorescent monomer-acid monomer solution that is dosed into the reaction medium; or the one or more additional monomers can be present as an additional monomer solution that is dosed to the reaction medium concurrently with at least part of the dosing of either the fluorescent monomer-acid monomer solution or the initiator solution.

The polymerization reaction can be allowed to continue after dosing of all reactants to the aqueous reaction medium is complete.

As the fluorescent monomer is dosed to the reaction mixture, it is consumed as part of the polymerization reaction and therefore there exists an equilibrium concentration of fluorescent monomer in the reaction mixture. Depending on the solubility of the fluorescent monomer in the reaction medium, the equilibrium concentration of the fluorescent monomer can be less than 1000 ppm, or less than 200 ppm, or less than 100 ppm in the reaction mixture, if the solvent is water.

To optimize the polymerization of the water-soluble fluorescent water polymer, it is preferred that the fluorescent monomer-acid monomer solution be dosed slowly into the reaction medium. Dosing of the fluorescent monomer-acid monomer solution is carried out over a time period of from about five minutes to about 24 hours; or from about 30 minutes to about 18 hours, or from about 1 hour to about ten hours. In one embodiment, the fluorescent monomer-acid monomer solution can be added at a rate of no more than 50% of the total dosage amount per hour, or no more than 40% of the total dosage amount per hour, or no more than 30% of the total dosage amount per hour, or no more than 25% of the total dosage amount per hour, or no more than 20% of the total dosage amount per hour, or no more than 15% of the total dosage amount per hour, or no more than 10% of the total dosage amount per hour.

In one embodiment the polymerization initiator solution is dosed to the reaction medium at a rate no faster than the rate of the dosage of the fluorescent monomer-acid monomer solution, based on the total dosage amount of polymerization initiator.

The skilled artisan will adjust the dosage rates and time of the reaction to achieve optimum polymerization of the water-soluble fluorescent water treatment polymer, based on the disclosure herein, taking into consideration the quantity of reactants, the visual appearance of the reaction mixture and the capacity and features of the reaction vessel and dosing apparatus used for each use of the disclosed method as well as the conversion of the fluorescent monomer to polymer during the polymerization process. For example, if the reaction mixture is cloudy, it indicates that the dosing rate needs to be decreased.

The reaction mixture typically is heated during the step of dosing of the reactants. The heating may be continued during the polymerization reaction until the reaction is substantially complete. In one embodiment the reaction may be terminated by discontinuing the heating of the reaction mixture. In one embodiment, if a co-solvent is used, the reaction may be terminated by distilling the co-solvent. The reaction temperature can be at least 30° C., 50° C., or at least 60° C., or at least 70° C., or at least 80° C. In one embodiment the polymerization reaction mixture is heated to its reflux temperature. In one embodiment the reaction temperature is in the range of 90-95° C.

Method B—Dosing of Initiator

In some water treatment polymers, the non-fluorescent monomers may have polymerization reactivities more similar to those of the selected fluorescent monomers. For example itaconic acid and maleic acid both have slower polymerization rates than acrylic acid and methacrylic acid. When itaconic acid or maleic acid or their salts or anhydrides are used as all or part of the carboxylic acid monomer, then it is possible to have either the carboxylic acid monomer or the fluorescent monomer, or both, present in their full amounts in the reaction medium at initiation of the polymerization reaction. The reaction rate is then controlled by the rate of dosing of the initiator to the reaction medium.

This method for polymerization of a water-soluble fluorescent water treatment polymer comprising one or more fluorescent coumarin, fluorescein, rhodamine, or Nile blue derivative monomer comprises the steps of
a) providing a quantity of fluorescent coumarin, fluorescein, rhodamine, or Nile blue derivative monomer,
b) adding the full amount of the fluorescent coumarin, fluorescein, rhodamine, or Nile blue derivative monomer and the carboxylic acid monomer into a reaction medium,
c) providing an initiator solution,
d) dosing said initiator solution to said reaction medium to initiate the polymerization reaction, and
e) maintaining the dosing of the initiator solution into the reaction medium during the polymerization reaction, such that the polymerization reaction continues while the initiator solution is being dosed to the reaction medium,
   wherein the polymerization reaction yields a water-soluble fluorescent polymer suitable for use in treatment of an industrial water system.

The fluorescent monomer can be added to the reaction medium as a solid and dissolved in the reaction medium, or the fluorescent monomer can first be dissolved in an appropriate solvent and then added to the reaction medium.

Method C—Dosing of Initiator and Fluorescent Monomer

In another embodiment, the polymerization comprises the steps of
a) dissolving a carboxylic acid monomer in a reaction medium,
b) providing a quantity of fluorescent coumarin, fluorescein, rhodamine, or Nile blue derivative monomer,
c) providing an initiator solution,
d) dosing said initiator solution to the reaction medium, and
e) dosing the fluorescent monomer to the reaction medium during the dosing of the initiator solution,
   wherein the polymerization reaction yields a water-soluble fluorescent polymer suitable for use in treatment of an industrial water system.

In this method, the reaction medium can be aqueous or non-aqueous. The fluorescent monomer can be added in the form of a solution or a solid. This polymerization method is useful when the carboxylic acid monomer is a relatively slow-reacting monomer, such as itaconic acid, maleic acid, or their anhydrides or salts.

In any of the foregoing polymerization methods, the product is an aqueous composition of the water-soluble fluorescent water treatment. In one embodiment the reaction product is an aqueous solution of the water-soluble treatment polymer in which the polymer is present as at least 10 wt %, in one embodiment at least 20 wt %, in one embodiment at least 30 wt %, in one embodiment at least 40 wt %. As an optional additional step the polymerization reaction product can be dried to a powder or granule.

In any of the foregoing polymerization methods, the polymerization initiators are any initiator or initiating system capable of liberating free radicals under the reaction conditions employed. The free radical initiators are present in an amount ranging from about 0.01% to about 3% by weight based on total monomer weight. In an embodiment, the initiating system is soluble in water to at least 0.1 weight percent at 25° C. Suitable initiators include, but are not limited to, peroxides, azo initiators as well as redox systems, such as erythorbic acid, and metal ion based initiating systems. Initiators may also include both inorganic and organic peroxides, such as hydrogen peroxide, benzoyl peroxide, acetyl peroxide, and lauryl peroxide; organic hydroperoxides, such as cumene hydroperoxide and t-butyl hydroperoxide. In an embodiment, the inorganic peroxides, such as sodium persulfate, potassium persulfate and ammonium persulfate, are preferred. In another embodiment, the initiators comprise metal ion based initiating systems including Fe and hydrogen peroxide, as well as Fe in combination with other peroxides. Organic peracids such as peracetic acid can be used. Peroxides and peracids can optionally be activated with reducing agents, such as sodium bisulfite, sodium formaldehyde, or ascorbic acid, transition metals, hydrazine, and the like. A preferred system is persulfate alone such as sodium or ammonium persulfate or a redox system with iron and persulfate with hydrogen peroxide. Azo initiators, especially water-soluble azo initiators, may also be used. Water-soluble azo initiators include, but are not limited to, 2,2′-Azobis[2-(2-imidazolin-2-yl)propane]dihydrochloride, 2,2′-Azobis[2-(2-imidazolin-2-yl)propane]disulfate dihydrate, 2,2′-Azobis(2-methylpropionamidine)dihydrochloride, 2,2′-Azobis[N-(2-carboxyethyl)-2-methylpropionamidine]hydrate, 2,2′-Azobis{2-[1-(2-hydroxyethyl)-2-imidazolin-2-yl]propane}dihydrochloride, 2,2′-Azobis[2-(2-imidazolin-2-yl)propane], 2,2′-Azobis(1-imino-1-pyrrolidino-2-ethylpropane)dihydrochloride, 2,2′-Azobis{2-methyl-N-[1,1-bis(hydroxymethyl)-2-hydroxyethyl]propionamide}, 2,2′-Azobis[2-methyl-N-(2-hydroxyethyl)propionamide] and others.

The molecular weight of the polymers may be controlled by various compounds used in the art including for example chain transfer agents such as mercaptans, ferric and cupric salts, bisulfites, and lower secondary alcohols, preferably isopropanol. The preferred weight average molecular weight is less than 50000, preferably less than 30000 and most preferably less than 20000. The preferred average molecular weight is greater than 1000, more preferably greater than 2000 and most preferably greater than 3000.

In one embodiment the resulting polymer solution can be neutralized to a desired pH with an appropriate base. The neutralization can occur before, during or after polymerization or a combination thereof. One skilled in the art will recognize that the dicarboxylic acid monomers are typically partially or completely neutralized before or during polymerization to increase reactivity of the monomers and improve their incorporation into the polymer. The polymers may be supplied as the acid or partially neutralized. This allows the water treatment formulator to formulate these polymers in low pH acidic formulations and high pH alkaline formulations.

Suitable neutralization agents include but are not limited to alkali or alkaline earth metal hydroxides, ammonia or amines. Neutralization agents can be sodium, potassium or ammonium hydroxides or mixtures thereof. Amines include but are not limited to ethanol amine, diethanolamine, triethanolamine and others.

While ammonia or amines can be utilized, in one embodiment the polymer is substantially free of ammonium or amine salts. Substantially free of ammonium or amine salts means that the acid groups in the polymer are neutralized with less than 10 mole percent ammonia or amine neutralizing agents, preferably less than 5 mole percent ammonia or amine neutralizing agents, more preferably less than 2 mole percent ammonia or amine neutralizing agents, and most preferably none at all. In another embodiment, ammonium or amine containing initiators, such as ammonium persulfate, or chain transfer systems are not utilized. Surprisingly, it has been found that the presence of ammonium or amine salts has a reduces the hypochlorite bleach stability of the polymer. The polymer is stable to hypochlorite bleach. In one embodiment, the polymer maintains hypochlorite bleach at pH 9 where more than half of the initial free chlorine is maintained after 1 hour at pH 9 at 25° C. in the presence of 10 ppm of active polymer.

### Water Treatment Polymers

Disclosed herein is a water-soluble fluorescent water treatment polymer made from a polymerization mixture comprising (i) one or more water-soluble carboxylic acid monomers or their salts or anhydrides, (ii) one or more non-quaternized fluorescent monomers.

Carboxylic Acid Monomers

Carboxylic acid monomers suitable for the water treatment polymers as disclosed herein include but are not limited to one or more of acrylic acid, methacrylic acid, maleic acid which can be derived from maleic anhydride, itaconic acid, citraconic acid, mesaconic acid, glutaconic acid, aconitic acid, ethacrylic acid, alpha-chloro-acrylic acid, alpha-cyano acrylic acid, alpha-chloro-methacrylic acid, alpha-cyano methacrylic acid, beta methyl-acrylic acid (crotonic acid), beta-acryloxy propionic acid, sorbic acid, alpha-chloro sorbic acid, angelic acid, tiglic acid, p-chloro cinnamic acid, any of their salts and anhydrides, and mixtures of any of the foregoing. In one embodiment the additional carboxylic acid monomers can include mono-alkylesters of dicarboxylic acids including maleic acid and fumaric acid, such as monomethyl maleate and monoethyl maleate.

In one embodiment, the carboxylic acid monomers include those which can dissolve the coumarin, fluorescein, rhodamine, or Nile blue derivative monomer, at any temperature from ambient up to the temperature at which the fluorescent monomer-acid monomer solution is dosed to the aqueous reaction medium, optionally in the presence of a co-solvent. Preferred carboxylic acid monomers for this purpose include acrylic acid and methacrylic acid, and combinations thereof, with acrylic acid being preferred.

Carboxylic acid monomers which are solid, such as maleic acid and itaconic acid, also can be used.

In one embodiment, the carboxylic acid monomers are water-soluble. As used herein with respect to water-soluble carboxylic acid monomers, water-soluble means that the monomer has a water solubility as the acid of greater than 1 gram per 100 mls of water at 25° C., preferably greater than 5 grams per 100 mls of water at 25° C., and most preferably greater than 10 grams per 100 mls of water at 25° C.

The total carboxylic acid monomers, including acrylic acid, methacrylic acid, maleic acid, itaconic acid and any additional carboxylic acid monomers, will be present in the polymerization mixture in the range of 10-99.9 mol %.

Fluorescent Monomers

The fluorescent monomers are coumarin, fluorescein, rhodamine, or Nile blue derivatives represented by the Structures (I), (III) and (V), wherein Structure (I) is provided above, and Structures (III) and (V) are provided below.

In one embodiment, the fluorescent monomer has the Structure III and is substantially free of the Structure IV, wherein Structure III is:

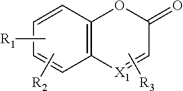

and Structure IV is:

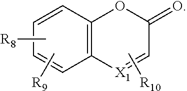

In another embodiment, the fluorescent monomer has the Structure III and is substantially free of the Structure IV, wherein:
R₁ is =NH₂+X⁻, =NH⁺alkyl X⁻, ═N⁺(alkyl)₂X⁻, H, (meth)acryloxy, vinylbenzyloxy, styryl, styryloxy, (meth)allyl, (meth)allyloxy, (meth)acryloxy polyethylene glycol, H—(CH₂CH₂O)ₙ₁—, ═O, OH, O⁻M⁺, CₙH₂ₙ₊₁CH═CHCH₂O—, CₙH₂ₙ₊₁CH═C(CH₃)CH₂O—, NH₂, NHalkyl, N(alkyl)₂, (meth)acrylamido, C₁-C₄alkyl, —COOH, —CH₂COOH, —COO⁻M⁺, —CH₂COO⁻M⁺, benzyl, CₙH₂ₙ₊₁CH═CHCH₂—, CₙH₂ₙ₊₁CH═C(CH₃)CH₂—, or oxyC₁₋₄ alkyl(meth) acrylates;
R₂ is H or CH₃;
R₃ is H, (meth)allyloxy, meth)allyloxy(CH₂CH₂O)ₙ₁—, (meth)acryloxy, (meth)acryloxy(CH₂CH₂O)ₙ₁—, H—(CH₂CH₂O)ₙ₁—, vinylbenzyl, vinylbenzyloxy, styryl, styryloxy, —OH, —O⁻M⁺, C₁-C₄alkyl, —COOH, —CH₂COOH, —COO⁻M⁺, —CH₂COO⁻M⁺, CH₃(CH₂)ₙ₂C(O)OCₙH₂ₙ₊₁CH═CHCH₂—, or CH₃(CH₂)ₙ₂C(O)O CₙH₂ₙ₊₁ CH═C(CH₃)CH₂—,
or R₃ is

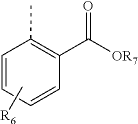

where the dotted bond joins R₃ to the remainder of Structure III;
R₆ is —NH₂, NHalkyl, N(alkyl)₂, (meth)acrylamido, (meth)allyl, —COOH, —COO⁻M⁺, (meth)acryloxy, styryl, styryloxy, vinylbenzyl, or H;
R₇ is H, M⁺, benzyl, CₙH₂ₙ₊₁CH═CHCH₂—, CₙH₂ₙ₊₁CH═C(CH₃)CH₂—, or oxyC₁₋₄ (meth)alkyl, CH₂═CR′—C(═O)—O—(CH₂)ₙ₁—O—C(═O)—O—;
R′ is H or CH₃;
with the proviso that R₁, R₃, R₆ or R₇ have at least one polymerizable double bond;
R₈ is ═NH₂, ═NHCH₂CH₃, ═N(CH₂CH₃)₂, H, ═O, OH, O⁻M⁺, NH₂, NHalkyl, N(alkyl)₂, C₁-C₄alkyl, —COOH, —CH₂COOH; —COO⁻M⁺, —CH₂COO⁻M⁺,
R₉ is H or CH₃;
R₁₀ is H, —OH, —O⁻M⁺, C₁-C₄alkyl, —COOH, —CH₂COOH, —COO⁻M⁺, or —CH₂COO⁻M⁺;
or R₁₀ is

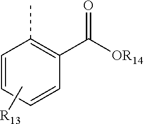

where the dotted bond joins R₁₀ to the remainder of Structure IV;
R₁₃ is —NH₂, —COOH, —COO⁻M⁺, or H;
R₁₄ is H or M⁺;
n is 0-10;
m is 2 when n is 0; and m is 1 when n is 0-10;
M⁺ is a cationic counterion; and
n1 is 2-4.

In another embodiment, the fluorescent monomer has the Structure IIIC and is substantially free of Structure IVC, wherein Structure IIIC is:

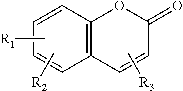

and Structure IVC is:

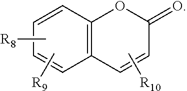

In one embodiment, the fluorescent monomer has the Structure V and is substantially free of the Structure VI, wherein Structure V is:

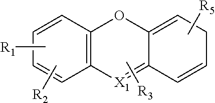

and Structure VI is:

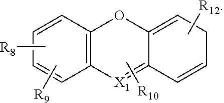

In one embodiment, the fluorescent monomer has the Structure V and is substantially free of the Structure VI, wherein:
R₁ is =NH₂+X⁻, =NH⁺alkyl X⁻, ═N⁺(alkyl)₂X⁻, H, (meth)acryloxy, vinylbenzyloxy, styryl, styryloxy, (meth)allyl, (meth)allyloxy, (meth)acryloxypolyethylene oxide, H—(CH₂CH₂O)ₙ₁—, ═O, OH, O⁻M⁺, CₙH₂ₙ₊₁CH═CHCH₂O—, CₙH₂ₙ₊₁CH═C(CH₃)CH₂O—, NH₂, NHalkyl, N(alkyl)₂, (meth)acrylamido, C₁-C₄alkyl, —COOH, —CH₂COOH, —COO⁻M⁺, —CH₂COO⁻M⁺, benzyl, CₙH₂ₙ₊₁CH═CHCH₂—, CₙH₂ₙ₊₁CH═C(CH₃)CH₂—, or oxyC₁₋₄ alkyl(meth)acrylates;
R₂ is H or CH₃;
R₃ is H, (meth)allyl oxy, meth)allyloxy(CH₂CH₂O)ₙ₁—, (meth)acryloxy, (meth)acryloxy(CH₂CH₂O)ₙ₁—, H—(CH₂CH₂O)ₙ₁—, vinylbenzyl, vinylbenzyloxy, styryl, styryloxy, —OH, —O⁻M⁺, C₁-C₄alkyl, —COOH, —CH₂COOH, —COO⁻M⁺, —CH₂COO⁻M⁺, CH₃(CH₂)ₙ₂C(O)OCₙH₂ₙ₊₁CH═CHCH₂—, or CH₃(CH₂)ₙ₂C(O)O CₙH₂ₙ₊₁ CH═C(CH₃)CH₂—,
or R₃ is

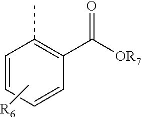

where the dotted bond joins R₃ to the remainder of Structure V;
R₅ is =NH₂+X⁻, ═NH⁺CH₂CH₃X⁻, ═N⁺(CH₂CH₃)₂X⁻, or ═O;
R₆ is —NH₂, (meth)acrylamido, (meth)allyl, —COOH, —COO⁻M⁺, (meth)acryloxy, styryl, styryloxy, vinylbenzyl, or H;
R₇ is H, M⁺, benzyl, CₙH₂ₙ₊₁CH═CHCH₂—, CₙH₂ₙ₊₁CH═C(CH₃)CH₂—, or oxyC₁₋₄ (meth)alkyl, CH₂═CR′—C(═O)—O—(CH₂)ₙ₁—O—C(═O)—O—;
R′ is H or CH₃;
with the proviso that R₁, R₃, R₆ or R₇ have at least one polymerizable double bond;
R₈ is ═NH₂, ═NHCH₂CH₃, ═N(CH₂CH₃)₂, H, ═O, OH, O⁻M⁺, NH₂, C₁-C₄alkyl, —COOH, —CH₂COOH; —COO⁻M⁺, —CH₂COO⁻M⁺,
R₉ is H or CH₃;
R₁₀ is H, —OH, —O⁻M⁺, C₁-C₄alkyl, —COOH, —CH₂COOH, —COO⁻M⁺, or —CH₂COO⁻M⁺;
or R₁₀ is

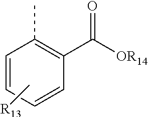

where the dotted bond joins R₁₀ to the remainder of Structure VI;
R₁₂ is =NH₂+X⁻, ═NH⁺CH₂CH₃X⁻, ═N⁺(CH₂CH₃)₂X⁻, or ═O;
R₁₃ is —NH₂, —COOH, —COO⁻M⁺, or H;
R₁₄ is H or M⁺;
n is 0-10;
m is 2 when n is 0; and m is 1 when n is 0-10;
M⁺ is a cationic counterion; and
n1 is 2-4.

In one embodiment, the fluorescent monomer has the Structure VC and is substantially free of the Structure VIC, wherein Structure VC is:

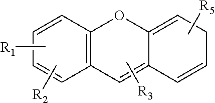

and Structure VIC is:

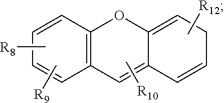

and the variables are as defined immediately above.

In one embodiment, the fluorescent monomer has the Structure VC-1 and is substantially free of the Structure VIC-1, wherein Structure VC-1 is:

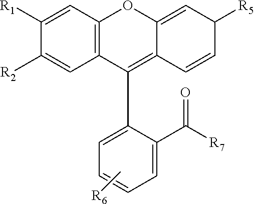

and Structure VIC-1 is:

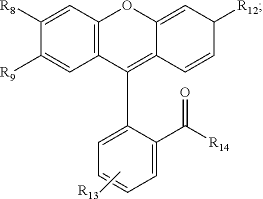

and the variables are as defined immediately above.

In one embodiment, the fluorescent monomer has the Structure VC-2 and is substantially free of the Structure VIC-2, wherein VC-2 is:

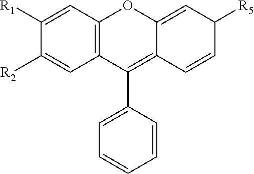

### and Structure VCI-2 is:

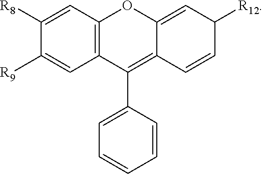

In one embodiment, the fluorescent monomer has the Structure VII and is substantially free of Structure VIII, wherein Structure VII is:

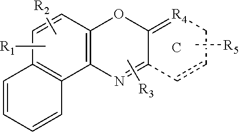

### and Structure VIII is:

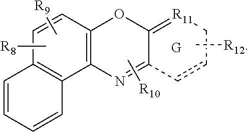

In one embodiment, the fluorescent monomer has the Structure VII-1 and is substantially free of the Structure VIII-1, wherein Structure VII-1 is:

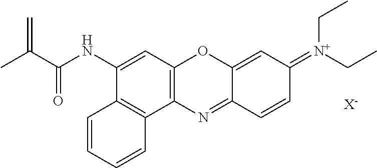

### and Structure VIII-1 is:

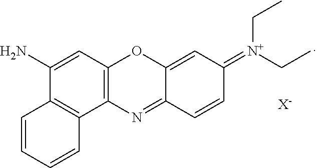

As used herein, unless otherwise indicated, “alkyl” groups, whether alone or a part of other groups, for example, “alkoxy” or “alkylene,” have any suitable carbon atom range, but preferably have 1-10 carbon atoms, most preferably 1-6 carbon atoms, and are optionally substituted by suitable substituents.

As used herein, unless otherwise indicated, “aryl” groups, whether alone or a part of other groups, for example, “aryloxy” or “arylalkoxy,” have any suitable carbon atom range, but preferably have 6-14 carbon atoms, most preferably 6 or 10 carbon atoms, i.e., phenyl or naphthyl, and are optionally substituted by suitable substituents.

As used herein, unless otherwise indicated, “suitable substituents” include, but are not limited to, halogen, such as F, Cl, Br or I; NO₂; CN; haloalkyl, typically CF₃; OH; amino; SH; —CHO; —CO₂H; oxo (═O); —C(═O)amino; NRC(═O)R; aliphatic, typically alkyl, particularly methyl; heteroaliphatic; —OR, typically methoxy; —SR; —S(═O)R; —SO₂R; aryl; or heteroaryl; where each R independently is aliphatic, typically alkyl, aryl, or heteroaliphatic. In certain aspects the optional substituents may themselves be further substituted with one or more unsubstituted substituents selected from the above list. Exemplary optional substituents include, but are not limited to: —OH, oxo (═O), —Cl, —F, Br, C₁₋₄alkyl, phenyl, benzyl, —NH₂, —NH(C₁₋₄alkyl), —N(C₁₋₄alkyl)₂, —NO₂, —S(C₁₋₄alkyl), —SO₂(C₁₋₄alkyl), —CO₂(C₁₋₄alkyl), and —O(C₁₋₄alkyl).

Unreacted fluorescent monomer present in the polymer formulation added to an industrial water system can result in an inaccurate measurement of the polymer in the aqueous system. In one aspect, the residual amount of unreacted fluorescent monomer in the polymerization reaction product is less than 15 mole percent of the fluorescent monomer added to the polymer, preferably less than 10 mole percent of the fluorescent monomer added to the polymer, preferably less than 5 mole percent of the fluorescent monomer added to the polymer, preferably less than 2.5 mole percent of the fluorescent monomer added to the polymer, and most preferably less than 1 mole percent of the fluorescent monomer added to the polymer.

Phosphorous-Containing Moieties

Optional phosphorus-containing moieties that can be incorporated into the polymer may be derived from any one or more of polymerizable phosphonate-containing monomers, phosphinic acid, phosphinate groups, phosphonic acid or phosphonate groups.

Polymerizable phosphonate monomers include without limitation vinyl phosphonic acid and vinyl diphosphonic acid, isopropenyl phosphonic acid, isopropenyl phosphonic anhydride, (meth)allylphosphonic acid, ethylidene diphosphonic acid, vinylbenzylphosphonic acid, 2-(meth)acrylamido-2-methylpropyl phosphonic acid, 3-(meth)acrylamido-2-hydroxypropylphosphonic acid, 2-(meth)acrylamidoethylphosphonic acid, benzyl phosphonic acid esters and 3-(meth)allyloxy-2-hydroxypropylphosphonic acid.

Phosphinic acid or phosphinate groups may be incorporated in the polymer as phosphino groups by including in the polymerization mixture molecules having the structure

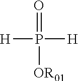

where R₀₁ is H, C₁-C₄ alkyl, phenyl, alkali metal or an equivalent of an alkaline earth metal atom, an ammonium ion or an amine residue. These moieties, which can incorporate phosphinic or phosphinate groups into the polymer, include but are not limited to hypophosphorous acid and its salts, such as sodium hypophosphite.

Phosphonic acid or phosphonate groups may be incorporated in the polymer by including in the polymerization mixture molecules having the structure

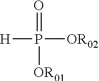

where R₀₁ or R₀₂ are independently H, C₁-C₄ alkyl, phenyl, alkali metal or an equivalent of an alkaline earth metal atom, an ammonium ion or an amine residue. These moieties include but are not limited to orthophosphorous acid and its salts and derivatives such as dimethyl phosphite, diethyl phosphite and diphenyl phosphite.

The one or more phosphorous moieties may be present in the water treatment polymer in the range of no greater than 20 mol %; in another aspect no greater than 10 mol %, in still another aspect no greater than 5 mol %, in still another aspect no greater than 3 mol %, and may not be present.

Sulfonic Acid Monomers

Optional water-soluble sulfonic acid monomers include but are not limited to one or more of 2-acrylamido-2-methyl propane sulfonic acid (‘AMPS’), vinyl sulfonic acid, sodium (meth)allyl sulfonate, sulfonated styrene, (meth)allyloxybenzene sulfonic acid, sodium 1-(meth allyloxy 2 hydroxy propyl sulfonate, (meth)allyloxy polyalkoxy sulfonic acid, (meth)allyloxy polyethoxy sulfonic acid and combinations thereof, and their salts. In various embodiments the sulfonic acid monomers can be present in the aqueous reaction medium before dosing of the fluorescent monomer-acid monomer solution begins, or can be mixed into the fluorescent monomer-acid monomer solution, or can be dosed into the polymerization mixture concurrently as a separate stream. In an embodiment, the sulfonic acid group can be incorporated in the polymer after polymerization. Examples of this type of sulfonic acid groups are sulfomethylacrylamide and sulfoethylacrylamide. For example, when the polymer contains acrylamide, the acrylamide moiety can react with formaldehyde and methanol to form sulfomethylacylamide.

In one embodiment, the amount of sulfonic acid monomer is less than 60 mole percent of the polymer, more preferably less than 40 mole percent of the polymer, more preferably less than 20 mole percent of the polymer and most preferably less than 10 mole percent of the polymer, and may not be present.

Nonionic Monomers

For purposes of this disclosure, a nonionic monomer is defined as a monomer not capable of developing a charge in water at any pH range. Non-ionic monomers suitable for use herein are preferably substantially free of amine groups. Nonionic monomers include water-soluble non-ionic monomers and low water solubility non-ionic monomers. The low water solubility non-ionic monomers are preferred.

As used herein with respect to water-soluble non-ionic monomers, water-soluble means that the monomer has a water solubility of greater than 6 grams per 100 mls of water at 25° C.

Examples of water-soluble non-ionic monomers include (meth)acrylamide, N,N-dimethylacrylamide, acrylonitrile, hydroxy alkyl (meth)acrylates such as hydroxyethyl (meth)acrylate and hydroxypropyl (meth)acrylate, vinyl alcohol typically derived from the hydrolysis of already polymerized vinyl acetate groups, ethoxylated (meth)allyl alcohol, (poly)alkoxylated (meth)acrylates such as poly(ethylene glycol)ₙ (meth)acrylate where n=1 to 100, preferably 3-50, and most preferably 5-20, ethoxylated alkyl, alkaryl or aryl monomers such as methoxypolyethylene glycol (meth)acrylate, 1-vinyl-2-pyrrolidone, vinyl lactam, allyl glycidyl ether, (meth)allyl alcohol, and others.

In one embodiment, the nonionic monomer is a low water solubility nonionic monomer which is defined as a nonionic monomer that has a water solubility of less than 6 g per 100 mls at 25° C., preferably less than 3 g per 100 mls at 25° C.

Examples of a low water solubility nonionic monomer include but are not limited to C₁-C₁₈ alkyl esters, C₂-C₁₈ alkyl-substituted (meth)acrylamides, aromatic monomers, alpha-olefins, C₁-C₆ alkyl diesters of maleic acid and itaconic acid, vinyl acetate, glycidyl methacrylate, (meth)acrylonitrile and others. C₁-C₁₈ alkyl esters of (meth)acrylic acid include but are not limited to methyl methacrylate, methyl acrylate, ethyl acrylate, n-butyl acrylate, n-butyl methacrylate, t-butyl acrylate and t-butyl methacrylate, 2-ethylhexyl (meth)acrylates, lauryl (meth)acrylate, stearyl (meth)acrylate and others. C₂-C₁₈ alkyl-substituted (meth)acrylamides include but are not limited to such as N,N-diethyl acrylamide, t-butyl acrylamide, and t-octyl acrylamide, and others. Aromatic monomers include but are not limited to styrene, alpha methylstyrene, benzyl (meth)acrylate and others. alpha-olefins include, propene, 1-butene, di isobutylene, 1 hexene and others. Preferred nonionic low water solubility monomers include styrene, methyl (meth)acrylate, di isobutylene, vinyl acetate, t-butyl acrylamide and ethyl acrylate.

In one embodiment, the amount of water-soluble nonionic monomer is no greater than 75 mole percent of the polymer, or no greater than 50 mole percent of the polymer, or no greater than 30 mole percent of the polymer, or may not be present.

In one embodiment, the amount of low water solubility nonionic monomer is no greater than 50 mole percent of the polymer, or no greater than 20 mole percent of the polymer, or no greater than 15 mole percent of the polymer, or no greater than 10 mole percent of the polymer or may not be present.

In one embodiment of the polymerization method as disclosed herein, water-soluble nonionic monomers can be present in the aqueous reaction medium before dosing of the fluorescent monomer-acid monomer solution begins.

In one embodiment of the polymerization method as disclosed herein, low water solubility nonionic monomers can be mixed into the fluorescent monomer-acid monomer solution before it is dosed to the aqueous reaction medium.

In one embodiment of the polymerization method as disclosed herein, any of the nonionic monomers can be dosed to the aqueous reaction medium as a separate dosing stream concurrently with the dosing of the fluorescent monomer-acid monomer solution.

### Fluorescent Monomer Compositions

Advantageously, the coumarin, fluorescein, rhodamine, and Nile blue derivative monomers used herein are soluble in compositions of acrylic acid or methacrylic acid that are essentially water free. This allows for the preparation of fluorescent monomer-acid monomer solutions that can be used as feed streams for the polymerization reaction to make the desired fluorescent water treatment polymers.

In another aspect, it is possible to have solutions of the low water-soluble fluorescent monomers as disclosed herein in solutions of acrylic acid or methacrylic acid or mixtures thereof, wherein the fluorescent monomer is present at a concentration higher than would be used in a polymerization reaction. Such solutions would facilitate ease of handling and storage of the fluorescent monomers prior to their use in a polymerization reaction, and could then be diluted with additional acid monomer and optionally other additional monomers to prepare monomer feed streams for the polymerization reaction in accordance with the method as disclosed herein. Such concentrated solutions could include at least 2 wt % fluorescent coumarin, fluorescein, rhodamine, or Nile blue derivative monomer, or at least 4 wt % fluorescent coumarin, fluorescein, rhodamine, or Nile blue derivative monomer, or at least 6 wt % fluorescent coumarin, fluorescein, rhodamine, or Nile blue derivative monomer, or at least 8 wt % fluorescent coumarin, fluorescein, rhodamine, or Nile blue derivative monomer, or at least 10 wt % fluorescent coumarin, fluorescein, rhodamine, or Nile blue derivative monomer, in a fluorescent coumarin, fluorescein, rhodamine, or Nile blue derivative monomer-acid monomer solution, wherein the acid monomer is acrylic acid, methacrylic acid, or a mixture thereof. In one embodiment such concentrated fluorescent coumarin, fluorescein, rhodamine, or Nile blue derivative monomer solutions contain less than 10 wt % water, or less than 5 wt % water, or less than 1 wt % water, or contain no detectable water.

In one embodiment, the disclosure relates to a fluorescent monomer composition, suitable as a premix in a process for preparing the disclosed water-soluble fluorescent polymers, wherein the fluorescent monomer composition comprises:
(a) one or more fluorescent coumarin and fluorescein derivative monomers as described hereinabove; and
(b) a solvent comprising acrylic acid, methacrylic acid, or a mixture thereof,
wherein said composition comprises at least 2 wt % of said one or more fluorescent monomers.

In a preferred embodiment, the the fluorescent monomer is incorporated into the water treatment polymer to an extent that the unreacted fluorescent monomer is as low as possible or undetectable. The unreacted fluorescent monomer will give a false signal of the polymer and needs to be minimized or eliminated.

It is important to measure the amount of unreacted fluorescent monomer at the end of every polymerization reaction. It is important to take samples during the reaction and measure the unreacted fluorescent monomer over the reaction to ensure as even an incorporation of the fluorescent monomer as possible as well as ensuring minimum amount of unreacted fluorescent monomer. If the unreacted fluorescent monomer is higher than desired, it can be minimized in a number of ways. The feed rate of the fluorescent monomer relative to the other monomers needs to be adjusted to get even incorporation of the fluorescent monomer as well as make sure that the residual fluorescent monomer is minimized. If the fluorescent monomer concentration is increasing during the reaction, it means that the other monomers are preferably reacting with themselves. In that case shorten the fluorescent monomer feed time and/or lengthen the feed time of the other monomers. This gives the fluorescent monomer a better chance of reacting with the other (presumably more reactive) monomers. If however, the fluorescent monomer is being used up too quickly, the opposite needs to be done. In that case lengthen the fluorescent monomer feed time and/or shorten the feed time of the other monomers. This gives the fluorescent monomer a better chance of reacting with the other (presumably more less reactive) monomers.

One skilled in the art will realize that monomers such as acrylic acid or 2-acrylamido-2-methyl propane sulfonic acid are reactive and may leave unreacted fluorescent monomer especially if it has allylic groups. In this case, a part of the fluorescent monomer may be added to the charge and the other part fed by itself or with the other monomers or the monomers feed adjusted as detailed above.

In most cases, it is not preferred to have all of the fluorescent monomer in the initial charge. However, if both the fluorescent monomer as well as the other monomer are unreactive, then they both may go into the charge. Such is the case when the fluorescent monomer is allylic and the other monomer is unreactive such as maleic acid or allylic such as (meth)allyl sulfonate and others.

The initiator feed needs to be as long as the total monomer feed or may exceed the monomer feed by 15-30 minutes. Other ways to minimize the unreacted fluorescent monomer include but are not limited to increasing the temperature, increasing the concentration of the initiator relative to the total amount of monomer, or changing the type of initiator. In addition, the finding the optimum pH to react the fluorescent monomer may help. Adding a water miscible cosolvent such as glycols or an alcohol like an isopropyl alcohol will help especially if the unreacted fluorescent monomer contains an aromatic group.

In a preferred embodiment, the fluorescent monomer is incorporated into the water treatment polymer to an extent of at least 80%, at least 90%, more preferably at least 95%, more preferably at least 97%, more preferably at least 98%, more preferably at least 99% and most preferably is undetectable.

In another preferred embodiment, the fluorescent monomer has Structure (I) and has less than 10 mol %, 5 mol %, 3 mol %, or less than 2 mol %, in each case based on 100 mol % of Structure (I), of Structure (II); and the fluorescent monomer is incorporated into the water treatment polymer to an extent of at least 90%, 95%, 97%, or at least 98%, or at least 99%. In an especially preferred embodiment, the fluorescent monomer has Structure (I) and has less than 2 mol %, based on 100 mol % of Structure (I), of Structure (II); and the fluorescent monomer is incorporated into the water treatment polymer to an extent of at least 98%.

In another preferred embodiment, the fluorescent monomer has Structure (III) and has less than 10 mol %, 5 mol %, 3 mol %, or less than 2 mol %, in each case based on 100 mol % of Structure (III), of Structure (IV); and the fluorescent monomer is incorporated into the water treatment polymer to an extent of at least 90%, 95%, 97%, or at least 98%, or at least 99%. In an especially preferred embodiment, the fluorescent monomer has Structure (III) and has less than 2 mol %, based on 100 mol % of Structure (III), of Structure (IV); and the fluorescent monomer is incorporated into the water treatment polymer to an extent of at least 98%.

In another preferred embodiment, the fluorescent monomer has Structure (V) and has less than 10 mol %, 5 mol %, 3 mol %, or less than 2 mol %, in each case based on 100 mol % of Structure (V), of Structure (VI); and the fluorescent monomer is incorporated into the water treatment polymer to an extent of at least 90%, 95%, 97%, or at least 98%, or at least 99%. In an especially preferred embodiment, the fluorescent monomer has Structure (V) and has less than 2 mol %, based on 100 mol % of Structure (V), of Structure (VI); and the fluorescent monomer is incorporated into the water treatment polymer to an extent of at least 98%.

In another preferred embodiment, the fluorescent monomer has Structure (IIIC) and has less than 10 mol %, 5 mol %, 3 mol %, or less than 2 mol %, in each case based on 100 mol % of Structure (IIIC), of Structure (IVC); and the fluorescent monomer is incorporated into the water treatment polymer to an extent of at least 90%, 95%, 97%, or at least 98%, or at least 99%. In an especially preferred embodiment, the fluorescent monomer has Structure (IIIC) and has less than 2 mol %, based on 100 mol % of Structure (IIIC), of Structure (IVC); and the fluorescent monomer is incorporated into the water treatment polymer to an extent of at least 98%.

In another preferred embodiment, the fluorescent monomer has Structure (VC) and has less than 10 mol %, 5 mol %, 3 mol %, or less than 2 mol %, in each case based on 100 mol % of Structure (VC), of Structure (VIC); and the fluorescent monomer is incorporated into the water treatment polymer to an extent of at least 90%, 95%, 97%, or at least 98%, or at least 99%. In an especially preferred embodiment, the fluorescent monomer has Structure (VC) and has less than 2 mol %, based on 100 mol % of Structure (VC), of Structure (VIC); and the fluorescent monomer is incorporated into the water treatment polymer to an extent of at least 98%.

In another preferred embodiment, the fluorescent monomer has Structure (VC-1) and has less than 10 mol %, 5 mol %, 3 mol %, or less than 2 mol %, in each case based on 100 mol % of Structure (VC-1), of Structure (VIC-1); and the fluorescent monomer is incorporated into the water treatment polymer to an extent of at least 90%, 95%, 97%, or at least 98%, or at least 99%. In an especially preferred embodiment, the fluorescent monomer has Structure (VC-1) and has less than 2 mol %, based on 100 mol % of Structure (VC-1), of Structure (VIC-1); and the fluorescent monomer is incorporated into the water treatment polymer to an extent of at least 98%.

### Use of the Water Treatment Polymers to Inhibit Scale

It is an advantage of the method disclosed herein that the polymerization end product is an aqueous solution of a water-soluble fluorescent water treatment polymer.

The polymer compositions may be added to the industrial water systems or may be formulated into various water treatment formulations which may then be added to the industrial water systems. In certain industrial water systems where large volumes of water are continuously treated to maintain low levels of deposited matter, the polymers may be used at levels as low as 0.5 ppm (parts per million). The upper limit on the level of polymer used will be dependent upon the particular aqueous system to be treated. For example, when used to disperse particulate matter the polymer may be used at levels ranging from 0.5 ppm to 2,000 ppm. When used to inhibit the formation or deposition of mineral scale the polymer may be used at levels ranging from 0.5 ppm to 100 ppm, preferably from 3 ppm to 20 ppm, more preferably from 5 ppm to 10 ppm.

Once prepared, the water-soluble polymers can be incorporated into a water treatment formulation comprising about 10-25 wt % of the water-soluble polymer and optionally other water treatment chemicals. Water treatment formulations may contain other ingredients such as corrosion inhibitors. These corrosion inhibitors can inhibit corrosion of copper, steel, aluminum, or other metals that may be present in the water treatment system. Azoles are typically used in these water treatment formulations as copper corrosion inhibitors. The benzotriazole is typically formulated in acidic formulations. The tolyl triazole is formulated in alkaline formulations. If a corrosion inhibitor is used, the formulator will choose a pH range suitable for the selected corrosion inhibitor, to achieve the desired solubility of these azoles, in the selected pH ranges. One skilled in the art will recognize that other azoles or non azole-containing copper corrosion inhibitors may be used in combination with these polymers. In addition, corrosion inhibitors that inhibit corrosion of other metals also can be used.

The fluorescent emissions of the treated water system are then monitored. Such monitoring can be accomplished using known techniques as disclosed, for example, in U.S. Pat. Nos. 5,171,450, 5,986,030, and 6,280,635. Fluorescent monitoring such as in-line monitoring allows the user to monitor the amount of water treatment polymer used to mitigate carbonate scale in the aqueous system. As indicated above, the level of the fluorescent polymer utilized in the water treatment compositions will be determined by the treatment level desired for the particular aqueous system to be treated. Conventional water treatment compositions are known to those skilled in the art. Once created, the fluorescent water-soluble polymers can be used as scale inhibitors in any industrial water system where a scale inhibitor is needed.

The other monomers of the fluorescent water treatment polymers as disclosed herein can be selected to provide water treatment polymers that are effective in the inhibition of any one or more of carbonate scale, phosphate scale, silica scale, and sulfate scale. In one embodiment the water treatment polymer is used to inhibit carbonate scale. In one embodiment the water treatment polymer is used to inhibit phosphate scale. One skilled in the art of water treatment polymers will understand how to select the carboxylic acid monomer and the other monomers of the water treatment polymer to optimize scale inhibition depending on the type of scale present in the system being treated. In general, polymers containing carboxylic acid monomers with or without phosphorus groups are good for carbonate and sulfate scale. Polymers containing carboxylic acid and sulfonic acid and polymers containing carboxylic acid, sulfonic acid and nonionic monomers are good for phosphate scale.

One skilled in the art will recognize that the fluorescent water treatment polymers of the disclosed method can be used in formulations containing inert tracers. These tracers include but are not limited to, 2-naphthalene sulfonic acid, rhodamine, Fluorescein and 1,3,6,8-Pyrenetetrasulfonic acid, tetrasodium salt (PTSA). This allows for complete monitoring of the system as described in U.S. Pat. Nos. 5,171,450 and 6,280,635.

### Use of the Water Treatment Polymers to Suppress Corrosion

In addition to the formation of scale, industrial water systems are subject to corrosion. While, as previously discussed, the water treatment formulations contemplated may contain other ingredients such as corrosion inhibitors, the fluorescent water treatment polymers disclosed herein exhibit the abilities to suppression corrosion, to transport iron, and to withstand high temperatures. This makes them suitable in one preferred embodiment for use to suppress corrosion while also controlling scale formation in boiler water systems.

Boilers are used to heat water to make steam for a variety of purposes, for example, to generate electricity, to heat buildings, and to provide hot water. Typical boiler deposits include calcium phosphate, calcium carbonate, magnesium hydroxide, magnesium oxide, silica, alumina, iron hydroxides, and iron oxides. Boiler deposits can cause tube overheating and tube failure resulting in severe property damage and even death in the case of explosion. Accordingly, corrosion control prevents deposits and preserves material integrity.

Oxygen dissolved in the process water is a culprit causing severe corrosion. The dissolved oxygen reacts with available iron to form various corrosive iron hydroxide and iron oxide species. As a result, particularly large boiler systems comprise a deaerator in-line to reduce the concentration of dissolved oxygen and other gases to low levels where corrosion is minimized. In the absence of a deaerator or other protective implement, these corrosive iron hydroxide and iron oxide species can slowly crystallize and deposit as tubercles, narrowing tube diameters over time, leading to pressure buildup, overheating, and catastrophic system failure.

It has been discovered that the fluorescent water treatment polymers disclosed herein exhibit the ability to transport iron through the system so that the iron is not available to react with dissolved oxygen to form corrosive species. It has also been discovered that the fluorescent water treatment polymers disclosed herein have the ability to disperse calcium phosphate and other typical boiler deposits. It has further been found that the fluorescent water treatment polymers disclosed herein are stable and can hold a signal at temperatures of 80-115° C., pH ranges of 7-12 and for 0.5 to 4 hours which is the typical residence time in these systems. Some boiler systems do have a deaerator but instead have a hot water tank that is typically in the temperature range of 80-95 C. Other systems contain pre-boiler and deaerator sections typically operate and eject feedwater at approximately 105-110° C. Therefore, the thermal stability of the fluorescent water treatment polymers disclosed herein coupled with their iron transport and boiler deposit dispersibility attributes makes them ideally suited for addition to boiler systems optionally including hot water tanks or pre-boilers and deaerators.

For such purposes, the water-soluble polymers can be incorporated into boiler feedwater or other water systems at levels ranging from 0.5 ppm to 2,000 ppm, preferably from 0.5 ppm to 100 ppm, more preferably from 1 ppm to 10 ppm, even more preferably from 2 ppm to 5 ppm.

In the same manner as discussed above, the fluorescent emissions of the boiler feedwater or other water system are then monitored in a manner well-known in the art. Fluorescent monitoring such as in-line monitoring allows the user to monitor the amount of water treatment polymer used to mitigate corrosion in the aqueous system and also the amount of iron being transported through the system. Adjustments to process conditions can be made depending on the monitored results, including the timing of subsequent water treatment polymer additions and the amount of water treatment polymer to be added.

### Use of the Water Treatment Polymers for Flocculation

Polymers for flocculation are formed from at least one fluorescent coumarin, fluorescein, rhodamine, or Nile blue derivative monomer, as herein described. Polymers for flocculation are high molecular weight polymers that are non-ionic, anionic, cationic, or amphoteric. These polymers are used in mineral processing, industrial and municipal wastewater treatment, oil sand tailings dewatering, paper making, biotechnology and other areas. The typical molecular weights of these polymers are 1,000,000 or higher and preferably in the 10,000,000 molecular weight range. These polymers are typically produced in inverse emulsion systems which lends itself to producing high molecular weight polymers. Nonionic flocculants contain at least one water soluble non-ionic monomer, as described above. These nonionic flocculants are typically based on acrylamide monomers which are typically produced in inverse emulsion systems. Anionic flocculants have carboxylic acid or sulfonic acid monomers (described before) and are typically used to flocculate positively charged particles. These are typically homopolymers of acrylic acid or copolymers of acrylic acid with acrylamide.

Cationic Polymers for flocculation comprise at least one water soluble cationic ethylenically unsaturated monomer and/or at least one water soluble non-ionic monomer, as described above.

As used herein, the term “cationic ethylenically unsaturated monomer” means an ethylenically unsaturated monomer which is capable of developing a positive charge in an aqueous solution or always has a positive charge because it is quaternized. In an embodiment of the present disclosure, the cationic ethylenically unsaturated monomer has at least one amine functionality.

As used herein, the term “amine salt” means that the nitrogen atom of the amine functionality is covalently bonded to from one to three organic groups and is associated with an anion.

As used herein with respect to water soluble non-ionic or cationic monomers for flocculation or coagulation purposes, “water soluble” means that the monomer has a water solubility of greater than 6 grams per 100 mls of water at 25° C.

The cationic ethylenically unsaturated monomers include, but are not limited to, N,N dialkylaminoalkyl(meth)acrylate, N-alkylaminoalkyl(meth)acrylate, N,N dialkylaminoalkyl(meth)acrylamide and N-alkylaminoalkyl(meth)acrylamide, where the alkyl groups are independently C₁₋₁₈ linear, branched or cyclic moieties. Aromatic amine containing monomers such as vinyl pyridine may also be used. Furthermore, acyclic monomers such as vinyl formamide, vinyl acetamide and the like which generate amine moieties on hydrolysis may also be used. Preferably the cationic ethylenically unsaturated monomer is selected from one or more of N,N-dimethylaminoethyl methacrylate, tert-butylaminoethylmethacrylate, N,N-dimethylaminopropyl methacrylamide, 3-(dimethylamino)propyl methacrylate, 2-(dimethylamino)propane-2-yl methacrylate, 3-(dimethylamino)-2,2-dimethylpropyl methacrylate, 2-(dimethylamino)-2-methylpropyl methacrylate and 4-(dimethylamino)butyl methacrylate and mixtures thereof. The most preferred cationic ethylenically unsaturated monomers are N,N-dimethylaminoethyl methacrylate, tert-butylaminoethylmethacrylate and N,N-dimethylaminopropyl methacrylamide.

Examples of cationic ethylenically unsaturated monomers that are quaternized include but are not limited to: dimethylaminoethyl (meth)acrylate methyl chloride quaternary salt, dimethylaminoethyl (meth)acrylate benzyl chloride quaternary salt, dimethylaminoethyl (meth)acrylate methyl sulfate quaternary salt, dimethylamino propyl (meth)acrylamide methyl chloride quaternary salt, dimethylamino propyl (meth)acrylamide methyl sulfate quaternary salt, diallyl dimethyl ammonium chloride, (meth)acrylamidopropyl trimethyl ammonium chloride and others.

Examples of water soluble non-ionic monomers for this purpose include (meth)acrylamide, N,N dimethylacrylamide, N,N diethylacrylamide, N isopropylacrylamide, acrylonitrile, hydroxy alkyl (meth)acrylates such as hydroxyethyl (meth)acrylate and hydroxypropyl (meth)acrylate, vinyl alcohol typically derived from the hydrolysis of already polymerized vinyl acetate groups, 1-vinyl-2-pyrrolidone, vinyl lactam, allyl glycidyl ether, (meth)allyl alcohol, and others. The preferred monomer is (meth)acrylamide. High molecular weight polyacrylamide polymers are typically produced by inverse emulsion polymerization. The fluorescent monomers of this disclosure can be incorporated into these polymers by dissolving these monomers into the acrylamide aqueous phase of the polymerization process.

A preferred cationic flocculant is a copolymer of dimethylaminoethyl (meth)acrylate methyl chloride quaternary salt and acrylamide typically produced in an inverse emulsions system.

Amphoteric polymers will contain a positive and negative charge. These positive and negative charges can be on different monomers such as dimethylaminoethyl (meth)acrylate methyl chloride quaternary salt and acrylic acid or on the same monomer which are zwitterionic or betaine monomers. These zwitterionic or betaine monomers are well known in the art.

When the polymers are used for flocculation in a water treatment system, the method comprises the steps of:
(a) dosing the water system with the water treatment polymer; and
(b) monitoring the fluorescent signal emitted from the water treatment system.

### Use of the Water Treatment Polymers for Cleaning Applications

Polymers for cleaning applications are formed from at least one fluorescent coumarin, fluorescein, rhodamine, or Nile blue derivative monomer, as herein described. In one embodiment, the disclosure relates to a method for determining whether a given location has been cleaned comprising the steps of:
(a) applying the polymer to the location;
(b) cleaning the location at least once; and
(c) attempting to detect the presence of the fluorescent coumarin, fluorescein, rhodamine, or Nile blue derivative monomer remaining at the location after said cleaning, which, presence, if detected, indicates that additional cleaning is needed.

Ideally, if fluorescent coumarin, fluorescein, rhodamine, or Nile blue derivative monomer is detected as remaining at the location after cleaning, the location should be cleaned again as necessary until residual fluorescent coumarin, fluorescein, rhodamine, or Nile blue derivative monomer can no longer be detected, which failure to detect residual fluorescent coumarin, fluorescein, rhodamine, or Nile blue derivative monomer indicates the location is completely clean.

In one embodiment, the polymer is provided as a part of a film-forming composition that quickly dries on the surface to be cleaned, is transparent, and is easily removed, but not by incidental contact. The film deposited on the surface fluoresces under ultraviolet light due to the presence of the fluorescent coumarin, fluorescein, rhodamine, or Nile blue derivative monomer and can be easily visualized by inspection with a hand-held UV light emitting light source, such as a UV flashlight.

Suitable compositions and their preparation and use are described in US 2016/0002525, the entire contents of which are incorporated herein by reference. Typically, the composition will contain a solvent and a thickener. A ready-to-use formulation will in one embodiment contain from about 1 to about 30 wt. % of a fluorescent polymer; from about 60 to about 99 wt. % of a solvent; and from about 0.05 to about 1 wt. % of a thickener. Preferably, the ready to use composition comprises from about 4 to about 25 wt. % of a fluorescent polymer; from about 50 to about 95 wt. % of a solvent; and from about 0.1 to about 0.4 wt. % of a thickener. More preferably, the ready to use composition comprises from about 8 to about 16% of a fluorescent polymer; from about 67 to about 91 wt. % of a solvent; from about 0.1 to about 0.4 wt. % of the thickener; from about 0.1 to about 0.7 wt. % of a preservative; and an optional pH adjusting agent. The composition can also be formulated as a concentrate, in which case, the weight ratio of the fluorescent polymer to surfactant, fluorescent polymer to thickener, or other relative proportions of ingredients will remain the same as in the ready-to-use composition, but the composition will contain a lesser amount of solvent.

In one embodiment, the solvent is preferably selected from water, methanol, ethanol, n-propanol, isopropanol, n-butanol, 2-butanol, isobutanol, n-pentanol, amyl alcohol, 4-methyl-2-pentanol, 2-phenylethanol, n-hexanol, 2-ethylhexanol, benzyl alcohol, ethylene glycol, ethylene glycol phenyl ether, ethylene glycol mono-n-butyl ether acetate, propylene glycol, propylene glycol mono and dialkyl ethers, propylene glycol phenyl ether, propylene glycol diacetate, dipropylene glycol, dipropylene glycol mono and dialkyl ethers, tripropylene glycol mono and dialkyl ethers, 1,3-propanediol, 2-methyl-1,2-butanediol, 3-methyl-1,2-butanediol, glycerol, methyl formate, ethyl formate, n-propyl formate, isopropyl formate, n-butyl formate, methyl acetate, n-propyl acetate, isopropyl acetate, isobutyl acetate, methyl lactate, ethyl lactate, propyl lactate, dimethylformamide, n-propyl propionate, n-butyl propionate, n-pentyl propionate, amyl acetate, methyl ethyl ketone, methyl isobutyl ketone, diisobutyl ketone, ethylamine, ethanolamine, diethanolamine, formic acid, acetic acid, propanoic acid, butanoic acid, acetone, acetonitrile, acetaldehyde, dimethyl sulfoxide, tetrahydrofuran, or a mixture thereof.

In one especially preferred embodiment, the solvent comprises water. The water can be from any source, including deionized water, tap water, softened water, and combinations thereof. The amount of water in the composition ranges from about 40 to about 99 wt. %, preferably from about 60 to about 95 wt. %, and more preferably from about 70 to about 90 wt. %.

In one embodiment, the thickener is preferably selected from xanthan gum, guar gum, modified guar, a polysaccharide, pullulan, an alginate, a modified starch, hydroxypropyl cellulose, hydroxypropyl methyl cellulose, carboxymethyl cellulose, hydroxyethyl cellulose, hydrophobically modified hydroxyethyl cellulose, hydrophobically modified hydroxypropyl cellulose, a polyacrylate, a vinyl acetate/alcohol copolymer, casein, a urethane copolymer, dimethicone PEG-8 polyacrylate, poly (DL-lactic-co-glycolic acid), a polyethylene glycol, a polypropylene glycol, pectin, or a combination thereof.

The composition can also include surfactants, preservatives, pH adjusting agents, and combinations thereof.

### EXAMPLES—FLUORESCENT MONOMERS

### Example Monomer 1: Diallyl Fluorescein Ether-Ester

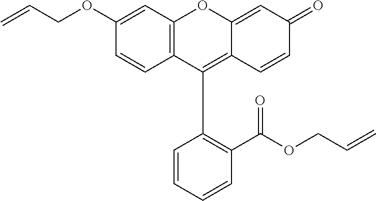 [SMILES: C=CCOC(=O)c1ccccc1-c1c2ccc(=O)cc-2oc2cc(OCC=C)ccc12]

Structure I comprises rings A and B and C:

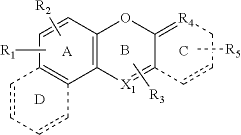

wherein
R₁ is allyloxy,
R₂ is H;
R₃ is

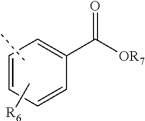

where the dotted bond joins R₃ to the remainder of Structure I;
X₁ is CH;
R₄ is CH and ring C is present;
R₅ is ═O;
R₆ is H;
R₇ is allyl;
and R₁ and R₇ have one polymerizable double bond;

A 250-mL Erlenmeyer flask was charged with 100 g of dimethylformamide (DMF) and 36.3 g (300 mmol) of allyl bromide. To this solution 37.6 g (100 mmol) of fluorescein disodium salt (uranine from Acros) powder was added. The deep red-brown suspension was stirred with a magnetic stir bar at ambient temperature in the capped flask and after about 20 min turned into a deep red solution. In another 40 min, solids precipitated out. 100 mL of DMF was added to allow stirring of the slightly warm mixture.

After stirring at ambient temperature for 48 hours, the mixture was poured into 800 mL of deionized water and extracted with 500 mL of ethyl acetate. Orange solids were present and a sizable rag layer separated the lower yellow-orange aqueous phase from the dark orange-brown ethyl acetate upper layer. After 20 minutes, the aqueous and rag layers were removed and the organic phase was washed with 100 mL of water, which was added to the aqueous phase. Two more extractions with 500 mL of ethyl acetate slightly improved separations, and each was counter-washed with 100 mL of water before combining with the first extraction. Rag layers were always added to the aqueous phase. A final 250-mL extraction with ethyl acetate gave a light orange solution. The pH of the aqueous layer was approximately 4-5 as measured by pH paper.

The combined organic phases were washed twice with 100 mL of water and once with 140 mL of brine before drying over 70 g of anhydrous Na₂SO₄ for 3 hr. Rotary evaporation of the decanted solution left 468 g of dark orange liquid, which was left at ambient temperature overnight. It was thick with crystals the next morning, and more ethyl acetate was stripped at 50° C. to give 401 g of mixture that was transferred to a 500 mL Erlenmeyer flask, heated to dissolve solids and left to crystallize.

The needles were suction filtered, washed with 100 mL of 3:1 ethyl acetate:hexanes and allowed to air dry to give 18.7 g of orange crystals melting point 153-156° C.

### HPLC/MS Procedure

2.6 mg of sample was dissolved in 1.0 of methanol and analyzed by LC/UVMS with listed conditions.

| | |
|---|---|
| Column | Agilent Porashell C8 4.6 mm × 50 mm |
| Solvents | 50% 25 mm AA pH 4.0 in 10% Acetonitrile |
| | 50% Acetonitrile |
| Flow Rate | 0.5 ml/min |
| Detection | UV 475 nm and MS Negative Ion Agilent 6550 QTOF |

### Quantitation Vs Fluorescein Standard

Fluorescein was not detected in the final product using the HPLC/MS procedure above. Therefore, Monomer Example 1 is free of Structure II which comprises rings E and F and G:

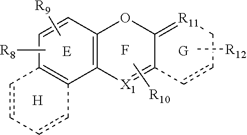

wherein R₈ is OH; R₉ is H;
R₁₀ is

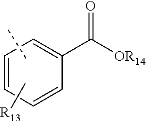

where the dotted bond joins R₁₀ to the remainder of Structure II; X₁ is CH;
R₁₁ is CH and ring G is present; R₁₂ is ═O; R₁₃ is H, R₁₄ is H.

### Monomer Example 2: Monoallyl Fluorescein Ether

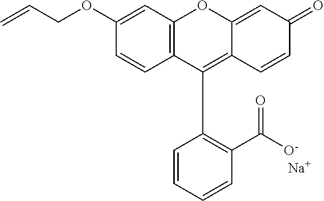 [SMILES: C=CCOc1ccc2c(-c3ccccc3C(=O)[O-])c3ccc(=O)cc-3oc2c1 ; [Na+]]

Structure I comprises rings A and B and C:

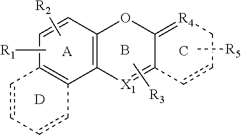

wherein
R₁ is allyloxy,
R₂ is H;
R₃ is

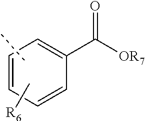

where the dotted bond joins R₃ to the remainder of Structure I;
X₁ is CH;
R₄ is CH and ring C is present;
R₅ is ═O;
R₆ is H;
R₇ is H;
and R₁ has one polymerizable double bond;

In a 500-mL Erlenmeyer flask 15.0 g (36.4 mmol) of diallyl fluorescein ether-ester (Monomer Example 1) was dissolved in 75 mL of hot tetrahydrofuran (THF) and then 2.91 g (36.4 mmol) of 50% NaOH was it added. The solution became thick with solids after 40 min of stirring on a “hot” plate.

The mixture was evaporated to a solid, which only partially dissolved in hot water, and the suspension was then filtered. The recovered solids were stirred in an aqueous suspension with another 1.38 g of 50% NaOH until nearly a complete solution and then filtered into the first solution.

The 300 mL of aqueous solution was stirred well at ambient temperature and was acidified with 5.4 g (54 mmol) of conc. HCl to give a slurry of orange solids. After filtration, the solids were dissolved in ˜140 g of ethanol, filtered quickly and allowed to recrystallize overnight.

The solid formed was filtered, washed with ethanol and then air dried to give ˜7 g of light yellow-brown crystals melting point 198-204° C.

### Analyses

Melting points were determined on a Fisher-Johns hot stage melting point apparatus.

### HPLC/MS Procedure

2.6 mg of sample was dissolved in 1.0 of methanol and analyzed by LC/UVMS with listed conditions.

| | |
|---|---|
| Column | Agilent Porashell C8 4.6 mm × 50 mm |
| Solvents | 50% 25 mm AA pH 4.0 in 10% Acetonitrile |
| | 50% Acetonitrile |
| Flow Rate | 0.5 ml/min |
| Detection | UV 475 nm and MS Negative Ion Agilent 6550 QTOF |

### Quantitation Vs Fluorescein Standard

Fluorescein (Structure II) was 9.8 area % of the final product as detected by the method above. Structure II comprises rings E and F and G:

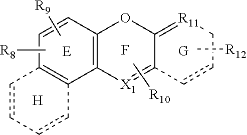

wherein R₈ is OH; R₉ is H;
R₁₀ is

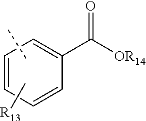

where the dotted bond joins R₁₀ to the remainder of Structure II; X₁ is CH;
R₁₁ is CH and ring G is present; R₁₂ is ═O; R₁₃ is H, R₁₄ is H.

### Monomer Example 3: 5-Acrylamidofluorescein

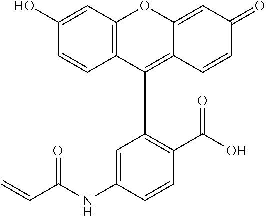 [SMILES: C=CC(=O)Nc1ccc(C(=O)O)c(-c2c3ccc(=O)cc-3oc3cc(O)ccc23)c1]

Structure I comprises rings A and B and C:

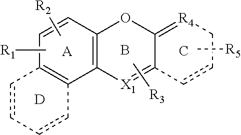

wherein R₁ is OH, R₂ is H;
R₃ is

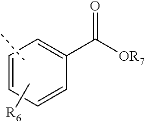

where the dotted bond joins R₃ to the remainder of Structure I;
X₁ is CH;
R₄ is CH and ring C is present; R₅ is ═O; R₆ is acrylamido; R₇ is H; and R₆ has one polymerizable double bond.

In a 100-mL, mechanically stirred 5-necked flask under nitrogen was placed 2.00 g (5.76 mmol) of 5-aminofluorescein (Acros) and 50 mL of acetone kept over 3 Å sieves to form a clear yellow-brown solution. This was cooled to −3° as 0.94 mL (1.04 g, 11.56 mmol, 2-fold excess) of acryloyl chloride was added dropwise via syringe. The first few drops caused the color to intensify to deep red, and then a precipitate began to form. The remaining acryloyl chloride was added over 7 min at −4° C. to −1° C. to give an orange slurry of solids that was allowed to warm to ambient temperature for 1 hr.

The solid was suction filtered in a 60-mL coarse frit funnel and washed well with acetone. Drying under high vacuum at ambient temperature for 3 hours gave 2.57 g of orange powder.

The product above was analyzed by LC/UV/MS using the procedure below:

About 1.0 mg of sample was dissolved in 1.0 mL of methanol and 200 μL of THF. This was analyzed by LC/UV/ELSD/MS with listed conditions.

| | |
|---|---|
| Column | Agilent C8 4 mm × 50 mm |
| Mobile Phase | A 25 mm AF pH 3.0 in 20% acetonitrile |
| | B Acetonitrile |
| Time 0 | 90% A/10% B |
| Time 5 | 50% A/50% B |
| Injection | 2 μL |
| Detection | UV 300 nm, Softa ELSD, Agilent 3550 QTOF Pos ion |

The desired product 5-Acrylamidofluorescein was 97.7 by area % and the starting material, 5-aminofluorescein was 1.2 area % of the final product as detected by LC/UV/ELSD/MS procedure above.

Monomer Example 3 is relatively free of aminofluorescein (Structure II):

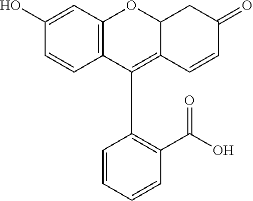 [SMILES: O=C1C=CC2=C(c3ccccc3C(=O)O)c3ccc(O)cc3OC2C1]

where Structure II comprises rings E and F and G:

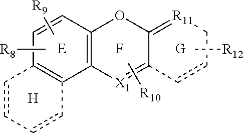

wherein R₈ is OH; R₉ is H;

### R₁₀ is

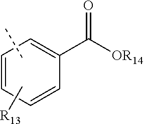

where the dotted bond joins R₁₀ to the remainder of Structure II; X₁ is CH; R₁₁ is CH and ring G is present; R₁₂ is ═O; R₁₃ is NH₂, R₁₄ is H.

### Monomer Example 4: 7-acryloxy-4-methylcoumarin

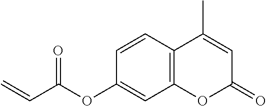 [SMILES: C=CC(=O)Oc1ccc2c(C)cc(=O)oc2c1]

In Monomer Example 4, Structure I has rings A and B:

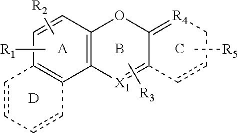

wherein
R₁ is acryloxy; R₂ is H; R₃ is alkyl (methyl);
X₁ is CH;
R₄ is O and ring C is absent;
and R₁ has a polymerizable double bond.

In a 50-mL 3-necked flask with magnetic stir bar was placed 0.52 g (12.5 mmol) of 97% NaOH flakes and 20.1 g of 200-proof ethanol (kept over anhydrous Na₂SO₄) under nitrogen. The mixture was stirred well until all caustic dissolved to give a light colored solution, and to this was then added 2.22 g (12.6 mmol) of 7-hydroxy-4-methylcoumarin (Acros 97%). The bright yellow suspension was cooled in ice as 1.13 mL (1.25 g, 13.9 mmol, 10 mol % excess) of acryloyl chloride began adding dropwise.

Within the first few drops the suspension became unstirrable, so we changed to a mechanical stirrer and continued addition over 22 min at 2-4° C. to give a very thick beige suspension. Stirring continued at 0-7° C. for 1 hour before the mixture was allowed to warm to room temperature and stand overnight and was swept with nitrogen evaporated almost all of the ethanol. We added 30 mL of water, filtered the solid, washed with more water and disposed of all aqueous portions.

The wet solid was heated with 60 g of methanol and was pressure filtered into a 125-mL Erlenmeyer flask to crystallize as white needles from the yellow solution. The remaining solid was transferred again to a flask and heated again with methanol, pressure filtered and the filtrate provided a little more solid. About 0.60 g of fluffy, long white needles, mp 150.5-151.5 was obtained.

### Analyses

### HPLC/UV/MS

The product above was analyzed by LC/UV/MS using the procedure below:

About 1.0 mg of sample was dissolved in 1.0 mL of methanol and 200 μL of THF. This was analyzed by LC/UV/ELSD/MS with listed conditions.

| | |
|---|---|
| Column | Agilent C8 4 mm × 50 mm |
| Mobile Phase | A 25 mm AF pH 3.0 in 20% acetonitrile |
| | B Acetonitrile |
| Time 0 | 90% A/10% B |
| Time 5 | 50% A/50% B |
| Injection | 2 μL |
| Detection | UV 300 nm, Softa ELSD, Agilent 3550 QTOF Pos ion |

The desired product 7-acryloxy-4-methylcoumarin was 97.7 by area % and the starting material, 7-hydroxy-4-methylcoumarin was 1.2 area %.
Therefore, Monomer Example 4 is relatively free of 7-hydroxy-4-methylcoumarin (Structure II)

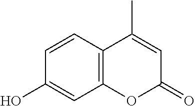

where Structure II comprises rings E and F:

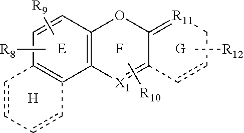

wherein
R₈ is OH; R₂ is H; R₃ is alkyl (methyl);
X₁ is CH;
R₄ is O and ring C is absent;

### EXAMPLES—FLUORESCENT POLYMERS

In the following Polymer Examples, stated quantities of fluorescent monomer are the quantities of the reaction products of the respective Monomer Examples.

### Polymer Example 1

An initial charge of 21.4 g of isopropyl alcohol mixed with 86.5 g of deionized water was added to a 1-liter glass reactor with inlet ports for an agitator, water cooled condenser, thermocouple, and adapters for the addition of monomer and initiator solutions. The mixture was heated to 84-85° C. A mixed monomer solution which consisted of 74.6 g of acrylic acid, 99.7 g of 2-acrylamido-2-methyl propane sulfonic acid sodium salt, 50% solution and 0.56 g of diallyl fluorescein ether-ester (monomer of Monomer Example 1, 0.11 mole % of polymer) was mixed and then fed to the reactor via measured slow-addition with stirring over a period of 3 hours. An initiator solution of 0.23 grams of sodium persulfate and 22 grams of water was concurrently added, starting at the same time as the monomer solution, for a period of 3.5 hours. The reaction product was then held at 85° C. for 60 minutes. The reactor was then set up for distillation. An azeotropic of 22 g of a mixture of water and isopropyl alcohol, was then distilled. 4.7 g of 50% sodium hydroxide was then added. The final polymer solution had a solids content of 20% and a pH of around 5. The conversion of diallyl fluorescein ether-ester monomer to polymer was expected to be greater than 95%.

The polymer sample was diluted in water to 10 ppm and the pH adjusted to 13 and the fluorescent signal was measured by using a Shimadzu RF-6000 model spectro fluorimeter. The excitation and emission wavelengths were 460 and 520 nm respectively and the signal strength was 3297. This high pH stability makes this polymer useful in boiler feed applications.

### Polymer Example 2

An initial charge of 21.4 g of isopropyl alcohol mixed with 86.5 g of deionized water was added to a 1-liter glass reactor with inlet ports for an agitator, water cooled condenser, thermocouple, and adapters for the addition of monomer and initiator solutions. The mixture was heated to 84-85° C. A mixed monomer solution which consisted of 74.6 g of acrylic acid, 99.7 g of 2-acrylamido-2-methyl propane sulfonic acid sodium salt, 50% solution and 0.56 g of allyl fluorescein (monomer of Monomer Example 2, 0.11 mole % of polymer) was mixed and then fed to the reactor via measured slow-addition with stirring over a period of 3 hours. An initiator solution of 0.23 grams of sodium persulfate and 22 grams of water was concurrently added, starting at the same time as the monomer solution, for a period of 3.5 hours. The reaction product was then held at 85° C. for 60 minutes. The reactor was then set up for distillation. An azeotropic of 22 g of a mixture of water and isopropyl alcohol, was then distilled. 4.7 g of 50% sodium hydroxide was then added. The final polymer solution had a solids content of 20% and a pH of around 5. The conversion of allyl fluorescein monomer to polymer was expected to be greater than 95%.

The polymer sample was diluted in water to 10 ppm and the pH adjusted to 13 and the fluorescent signal was measured by using a Shimadzu RF-6000 model spectro fluorimeter. The excitation and emission wavelengths were 460 and 520 nm respectively and the signal strength was 3297.

The fluorescent signal was measured in the presence of chlorine bleach at pH 7 and a phosphate buffer.

| | Fluorescent signal at 258 nanometers | |
|---|---|---|
| | excitation and 519 nanometers of | Chlorine |
| Sample | emission | (ppm) |
| Polymer example | 3995 | 0.86 |
| 2 at initial time | | |
| Polymer example | 3602 | 0.80 |
| 2 after 1 hour | | |

These data indicate that the fluorescent signal is stable in the presence of bleach. In addition, the hypochlorite bleach or chlorine is maintained in the presence of the tagged polymer. This is particularly important for polymers used in scale control. These polymers need to function in the presence of oxidizing biocides such as chlorine and stabilize bromine.

### Polymer Example 3

An initial charge of 12.1 g of isopropyl alcohol mixed with 66.8 g of deionized water was added to a glass reactor with inlet ports for an agitator, water cooled condenser, thermocouple, and adapters for the addition of monomer and initiator solutions. The mixture was heated to 84-85° C. A mixed monomer solution which consisted of 9.8 g of acrylic acid, 13.1 g of 2-acrylamido-2-methyl propane sulfonic acid sodium salt, 50% solution and 0.08 g of 7-acryloxy-4-methylcoumarin (monomer of Monomer Example 4) (0.2 mol % of polymer) was mixed and then fed to the reactor via measured slow-addition with stirring over a period of 3 hours. An initiator solution of 0.23 grams of sodium persulfate and 22 grams of water was concurrently added, starting at the same time as the monomer solution, for a period of 3.5 hours. The reaction product was then held at 85° C. for 60 minutes. The reactor was then set up for distillation. An azeotropic of 26 g of a mixture of water and isopropyl alcohol, was then distilled. 4.7 g of 50% sodium hydroxide was then added. The final polymer solution had a solids content of 20.0% and a pH of around 5. The 7-acryloxy-4-methylcoumarin monomer was undetected in the final product.

The polymer sample was diluted in water to 10 ppm and the pH adjusted to 9 and the fluorescent signal was measured by using a Shimadzu RF-6000 model spectro fluorimeter. The excitation and emission wavelengths were 350 and 450 nm respectively and the signal strength was 11274.

### Water Soluble Anionic and Nonionic Copolymers

### Example 4: Poly[acrylamide-co-ammonium Acrylate] Inverse Emulsion

A reaction flask is equipped with an overhead mechanical stirrer, thermometer, nitrogen sparge tube, and condenser is charged an oil phase of paraffin oil (135.0 g, Exxsol D80 oil, Exxon—Houston, Tex.) and surfactants (4.5 g Atlas G-946 and 9.0 g Hypermer® B246SF). The temperature of the oil phase is then adjusted to 37° C.

An aqueous phase is prepared separately which comprised 50-wt. % acrylamide solution in water (126.5 g), acrylic acid (68.7 g), deionized water (70.0 g), and 0.8 g of 5-Acrylamidofluorescein (monomer of Example 3) dissolved in 6 grams of water and Versene 100 chelant solution (0.7 g). The aqueous phase is then adjusted to pH 5.4 with the addition of ammonium hydroxide solution in water (33.1 g, 29.4-wt. % as NH₃). The temperature of the aqueous phase after neutralization is 39° C.

The aqueous phase is then charged to the oil phase while simultaneously mixing with a homogenizer to obtain a stable water-in-oil emulsion. This emulsion is then mixed with a 4-blade glass stirrer and sparged with nitrogen for 60 minutes. During the nitrogen sparge the temperature of the emulsion is adjusted to 50±1° C. The sparge is discontinued and a nitrogen blanket implemented.

The polymerization is initiated by feeding a 3-wt. % azobisisobutyronitrile/AIBN solution in toluene (0.213 g) over a period of 2 hours. During the course of the feed the batch temperature is allowed to exotherm to 62° C. (^{˜}50 minutes), after which the batch is maintained at 62±1° C. After the feed the batch is held at 62±1° C. for 1 hour. Afterwards 3-wt. % AIBN solution in toluene (0.085 g) is then added in under one minute. Then the batch is held at 62±1° C. for 2 hours. Then batch is then cooled to room temperature and the product collected.

### Example 5: Polyacrylamide Inverse Emulsion

A reaction flask is equipped with an overhead mechanical stirrer, thermometer, nitrogen sparge tube, and condenser is charged an oil phase of paraffin oil (135.0 g, Exxsol D80 oil, Exxon—Houston, Tex.) and surfactants (4.5 g Atlas G-946 and 9.0 g Hypermer® B246SF). The temperature of the oil phase is then adjusted to 37° C. An aqueous phase is prepared separately which comprised 50-wt. % acrylamide solution in water (180 g), deionized water (70.0 g), and 0.9 g of monoallyl fluorescein ether (monomer of Example 2) dissolved in 10 grams of water and Versene 100 chelant solution (0.7 g).

The aqueous phase is then charged to the oil phase while simultaneously mixing with a homogenizer to obtain a stable water-in-oil emulsion. This emulsion is mixed with a 4-blade glass stirrer and is sparged with nitrogen for 60 minutes. During the nitrogen sparge the temperature of the emulsion is adjusted to 50±1° C. The sparge is discontinued and a nitrogen blanket implemented.

The polymerization is initiated by feeding a 3-wt. % azobisisobutyronitrile (AIBN) solution in toluene (0.213 g) over a period of 2 hours. During the course of the feed the batch temperature is allowed to exotherm to 62° C. (^{˜}50 minutes), after which the batch is maintained at 62±1° C. After the feed the batch is held at 62±1° C. for 1 hour. This corresponds to a second AIBN charge as AIBN of 100 ppm on a total monomer basis. Then the batch is held at 62±1° C. for 2 hours. Then batch is then cooled to room temperature and the product collected.

### Example 6: Cationic Copolymers

A reaction flask is equipped with an overhead mechanical stirrer, thermometer, nitrogen sparge tube, and condenser is charged an oil phase of paraffin oil (139.72 g Exxsol® D80, Exxon, Houston, Tex.) and surfactants (4.66 g Atlas® G-946 and 9.32 g Hypermer® B246SF, Croda.). The temperature of the oil phase is then adjusted to 37° C.

An aqueous phase is prepared separately which comprised 53-wt. % acrylamide solution in water (115.76 g), [2-(acryloyloxy)ethyl]trimethyl ammonium chloride (AETAC) (56.0 g) (80% by weight solution), deionized water (88.69 g), and 0.75 g of 5-Acrylamidofluorescein (monomer of Example 3) dissolved in 6 grams of water and Versene 100 (Dow Chemical, Midland, Mich.) chelant solution (0.6 g).

The aqueous phase is then charged to the oil phase while simultaneously mixing with a homogenizer to obtain a stable water-in-oil emulsion. This emulsion is then mixed with a 4-blade glass stirrer while being sparged with nitrogen for 60 minutes. During the nitrogen sparge the temperature of the emulsion is adjusted to 50+1° C. The sparge is discontinued and a nitrogen blanket implemented.

The polymerization is initiated by feeding a 3-wt. % AIBN (0.12 g) solution in toluene (3.75 g) over a period of 2-hours. During the course of the feed the batch temperature is allowed to exotherm to 62° C. (^{˜}50 minutes), after which the batch is maintained at 62±1° C. for 1-hour. Afterwards 3-wt. % AIBN (0.05 g) solution in toluene (1.50 g) is then charged in one shot. Then the batch is held at 62±1° C. for 2-hour. Then batch is cooled to room temperature and the product collected.

### Polymer Example 7: Performance in Boiler Feed Water Applications

The polymers of Examples 1 and 2 were diluted in water to 5000 ppm and the pH adjusted to 11.5. 125 milligrams of sodium metabisulfite was added, and the polymers were aged at different temperatures to simulate a hot water tank or an deaerator. After cooling, the samples were diluted to 10 ppm, and the fluorescent signal was measured by using a Shimadzu RF-6000 model spectrophotometer fluorimeter at the excitation and emission wavelengths (λ) detailed below.

| | | Aging | | λ | λ | Fluorescent | Fluorescent |
|---|---|---|---|---|---|---|---|
| | | Temperature | Aging | excitation | emission | signal before | signal after |
| Example | Polymer | ° C. | (hours) | (nm) | (nm) | aging | aging |
| 7a | Polymer | 114 | 4 | 460 | 520 | 3811 | 4408 |
| | Example | | | | | | |
| | 1 | | | | | | |
| 7b | Polymer | 85 | 4 | 460 | 520 | 3915 | 4016 |
| | Example | | | | | | |
| | 1 | | | | | | |
| 7c | Polymer | 114 | 4 | 484 | 515 | 3917 | 4873 |
| | Example | | | | | | |
| | 2 | | | | | | |
| 7d | Polymer | 85 | 4 | 484 | 515 | 4305 | 4603 |
| | Example | | | | | | |
| | 2 | | | | | | |

These data indicate that the polymer signal does not drop after aging at these temperatures and time periods. Therefore, the polymer can be used in boiler applications that may use a hot water tank which typically operates in the temperature range 80-95° C. or a deaerator which may operate as high as 114° C.

1. A fluorescent monomer of Structure I which is substantially free of Structure II, wherein Structure I comprises rings A and B and optionally rings C and/or D: 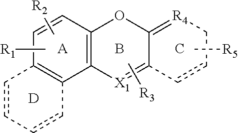
wherein
R₁ is H, alkyl, aryl, arylalkyl, aryloxy, NH₂, NHalkyl, N(alkyl)₂, =NH⁺alkyl X⁻, ═N(alkyl)₂X⁻, ═NH₂⁺X⁻, (meth)acryloyloxy, vinylaryl, vinylarylalkyl, vinylaryloxy, vinylarylalkyloxy, (meth)allyl, (meth)allyloxy, (meth)acryloxy, (meth)acryloxyalkyl, (meth)acryloxypolyalkylene oxide, (meth)allylamino, (meth)acrylamido, polyalkylene oxide, oxo, OH, O⁻M⁺, alkoxy, CₙH₂ₙ₊₁CH═CH-alkylene-, CₙH₂ₙ₊₁CH═C(CH₃)-alkylene-, CₙH₂ₙ₊₁CH═CH-alkylene-O—, CₙH₂ₙ₊₁CH═C(CH₃)-alkylene-O—, —COOH, -alkylene-COOH, —COO⁻M⁺, -alkylene-COO⁻M⁺, or —O-alkylene-(meth)acrylate;
R₂ is H or alkyl;
R₃ is H, alkyl, aryl, arylalkyl, aryloxy, (meth)allyl, (meth)allylamino, (meth)allyloxy, (meth)allyloxyalkoxide, (meth)acryloyloxy, ((meth)acryloxyalkyl, meth)acryloxypolyalkylene oxide, (meth)acryloyloxyalkoxide, polyalkylene oxide, vinylaryl, vinylarylalkyl, vinylaryloxy, vinylarylalkyloxy, —OH, or —O⁻M⁺, alkoxy, —COOH, -alkylene-COOH, —COO⁻M⁺, —CH₂COO⁻M⁺, CH₃(CH₂)ₙ₂C(O)OCₙH₂ₙ₊₁CH═CHCH₂—, CH₃(CH₂)ₙ₂C(O)O or CₙH₂ₙ₊₁CH═C(CH₃)CH₂—;
or R₃ is 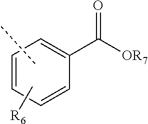
where the dotted bond joins R₃ to the remainder of Structure I;
X₁ represents CH or N;
R₄ is O when ring C is absent; or represents CH when ring C is present;
R₅ is ═NH₂⁺X⁻, =NH⁺alkyl X⁻, ═N⁺(alkyl)₂X⁻, NH₂, NHalkyl, N(alkyl)₂, or ═O;
R₆ is H, alkyl, alkylene, —NH₂, NHalkyl, N(alkyl)₂, (meth)acryloxy, (meth)acrylamido, (meth)allyl, —COOH, —COO⁻M⁺, phosphate, phosphonate, sulfonate, vinylaryl, vinylarylalkyl, vinylaryloxy, vinylarylalkyloxy, (meth)allyloxy, (meth)acryloxy, (meth)acryloxypolyalkylene oxide, (meth)acrylamido, polyalkylene oxide, (meth)acryloxyalkyl, or (meth)allylamino;
R₇ is H, M⁺, alkyl, alkenyl, arylalkyl, alkyl-CH═CH-alkylene-, alkyl-CH═C(CH₃)-alkylene-, —O-(meth)-alkyl, CH₂═CR′—C(═O)—O-alkylene-O—C(═O)—O—, (meth)allyloxy, (meth)acryloxy, (meth)acryloxypolyalkylene oxide, (meth)acrylamido, polyalkylene oxide, (meth)acryloxyalkyl, or (meth)allylamino;
R′ is H or alkyl;
n is 0-10;
m is 2 when n=0; and m is 1 when n=1-10;
X⁻ is an anionic counter ion; and
M⁺ is a cationic counterion;
with the proviso that R₁, R₃, R₆ or R₇ have at least one polymerizable double bond;
and Structure II comprises rings E and F and optionally rings G and/or H: 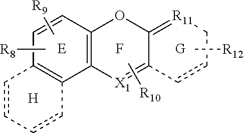
wherein
R₈ is H, alkyl, NH₂, NHalkyl, N(alkyl)₂, ═NH₂⁺X⁻, =NH⁺alkyl X⁻, ═N(alkyl)₂X⁻, ═O, OH, or O⁻M⁺, —COOH, -alkylene-COOH, or —COO⁻M⁺, or -alkylene-COO⁻M⁺;
R₉ is H or alkyl;
R₁₀ is H, —OH, or —O⁻M⁺, alkyl, —COOH, -alkylene-COOH, —COO⁻M⁺, or -alkylene-COO⁻M⁺;
or R₁₀ is 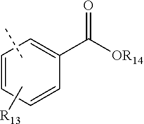
where the dotted bond joins R₁₀ to the remainder of Structure II;
X₁ is CH or N;
R₁₁ is O when ring G is absent; or represents CH when ring G is present;
R₁₂ is ═NH₂⁺X⁻, =NH⁺alkyl X⁻, ═N⁺(alkyl)₂X⁻, NH₂, NHalkyl, N(alkyl)₂, or ═O;
R₁₃ is H, —NH₂, NHalkyl, N(alkyl)₂, —COOH, —COO⁻M⁺, phosphate, phosphonate, or sulfonate;
R₁₄ is H or M⁺;
X⁻ has the meaning given above;
M⁺ has the meaning given above; and
rings D and H, when present, are optionally substituted.

2. The fluorescent monomer according to claim 1, which has the Structure III and is substantially free of the Structure IV, wherein Structure III is: 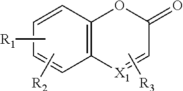
and Structure IV is:

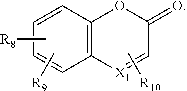

3. The fluorescent monomer according to claim 2, wherein:
R₁ is ═NH₂⁺X⁻, =NH⁺alkyl X⁻, ═N⁺(alkyl)₂X⁻, H, (meth)acryloxy, vinylbenzyloxy, styryl, styryloxy, (meth)allyl, (meth)allyloxy, (meth)acryloxy polyethylene glycol, H—(CH₂CH₂O)ₙ₁—, ═O, OH, O⁻M⁺, CₙH₂ₙ₊₁CH═CHCH₂O—, CₙH₂ₙ₊₁CH═C(CH₃)CH₂O—, NH₂, NHalkyl, N(alkyl)₂, (meth)acrylamido, C₁-C₄alkyl, —COOH, —CH₂COOH, —COO⁻M⁺, —CH₂COO⁻M⁺, benzyl, CₙH₂ₙ₊₁CH═CHCH₂—, CₙH₂ₙ₊₁CH═C(CH₃)CH₂—, or oxyC₁₋₄ alkyl(meth) acrylates;
R₂ is H or CH₃;
R₃ is H, (meth)allyloxy, meth)allyloxy(CH₂CH₂O)ₙ₁—, (meth)acryloxy, (meth)acryloxy(CH₂CH₂O)ₙ₁—, H—(CH₂CH₂O)ₙ₁—, vinylbenzyl, vinylbenzyloxy, styryl, styryloxy, —OH, —O⁻M⁺, C₁-C₄alkyl, —COOH, —CH₂COOH, —COO⁻M⁺, —CH₂COO⁻M⁺, CH₃(CH₂)ₙ₂C(O)OCₙH₂ₙ₊₁CH═CHCH₂—, or CH₃(CH₂)ₙ₂C(O)O CₙH₂ₙ₊₁CH═C(CH₃)CH₂—,
or R₃ is 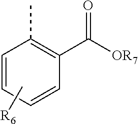
where the dotted bond joins R₃ to the remainder of Structure III;
R₆ is —NH₂, NHalkyl, N(alkyl)₂, (meth)acrylamido, (meth)allyl, —COOH, —COO⁻M⁺, (meth)acryloxy, styryl, styryloxy, vinylbenzyl, or H;
R₇ is H, M⁺, benzyl, CₙH₂ₙ₊₁CH═CHCH₂—, CₙH₂ₙ₊₁CH═C(CH₃)CH₂—, or oxyC₁₋₄ (meth)alkyl, CH₂═CR′—C(═O)—O—(CH₂)ₙ₁—O—C(═O)—O—;
R′ is H or CH₃;
with the proviso that R₁, R₃, R₆ or R₇ have at least one polymerizable double bond;
R₈ is ═NH₂, ═NHCH₂CH₃, ═N(CH₂CH₃)₂, H, ═O, OH, O⁻M⁺, NH₂, NHalkyl, N(alkyl)₂, C₁-C₄alkyl, —COOH, —CH₂COOH; —COO⁻M⁺, —CH₂COO⁻M⁺,
R₉ is H or CH₃;
R₁₀ is H, —OH, —O⁻M⁺, C₁-C₄alkyl, —COOH, —CH₂COOH, —COO⁻M⁺, or —CH₂COO⁻M⁺;
or R₁₀ is 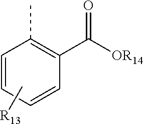
where the dotted bond joins R₁₀ to the remainder of Structure IV;
R₁₃ is —NH₂, —COOH, —COO⁻M⁺, or H;
R₁₄ is H or M⁺;
n is 0-10;
m is 2 when n is 0; and m is 1 when n is 0-10;
M⁺ is a cationic counterion; and
n1 is 2-4.

4. The fluorescent monomer according to claim 3, which has the Structure IIIC and is substantially free of Structure IVC, wherein Structure IIIC is: 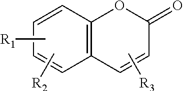
and Structure IVC is:

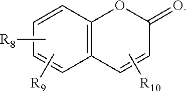

5. The fluorescent monomer according to claim 1, which has the Structure V and is substantially free of the Structure VI, wherein Structure V is: 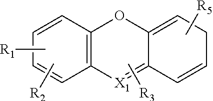
and Structure VI is:

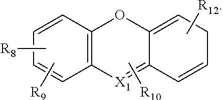

6. The fluorescent monomer according to claim 5, wherein:
R₁ is ═NH₂⁺X⁻, =NH⁺alkyl X⁻, ═N⁺(alkyl)₂X⁻, H, (meth)acryloxy, vinylbenzyloxy, styryl, styryloxy, (meth)allyl, (meth)allyloxy, (meth)acryloxypolyethylene oxide, H—(CH₂CH₂O)ₙ₁—, ═O, OH, O⁻M⁺, CₙH₂ₙ₊₁CH═CHCH₂O—, CₙH₂ₙ₊₁CH═C(CH₃)CH₂O—, NH₂, NHalkyl, N(alkyl)₂, (meth)acrylamido, C₁-C₄alkyl, —COOH, —CH₂COOH, —COO⁻M⁺, —CH₂COO⁻M⁺, benzyl, CₙH₂ₙ₊₁CH═CHCH₂—, CₙH₂ₙ₊₁CH═C(CH₃)CH₂—, or oxyC₁₋₄ alkyl(meth)acrylates;
R₂ is H or CH₃;
R₃ is H, (meth)allyl oxy, meth)allyloxy(CH₂CH₂O)ₙ₁—, (meth)acryloxy, (meth)acryloxy(CH₂CH₂O)ₙ₁—, H—(CH₂CH₂O)ₙ₁—, vinylbenzyl, vinylbenzyloxy, styryl, styryloxy, —OH, —O⁻M⁺, C₁-C₄alkyl, —COOH, —CH₂COOH, —COO⁻M, —CH₂COO⁻M⁺, CH₃(CH₂)ₙ₂C(O)OCₙH₂ₙ₊₁CH═CHCH₂—, or CH₃(CH₂)ₙ₂C(O)O CₙH₂ₙ₊₁CH═C(CH₃)CH₂—,
or R₃ is 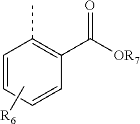
where the dotted bond joins R₃ to the remainder of Structure V;
R₅ is ═NH₂⁺X⁻, ═NH⁺CH₂CH₃X⁻, ═N⁺(CH₂CH₃)₂X⁻, or =0;
R₆ is —NH₂, (meth)acrylamido, (meth)allyl, —COOH, —COO⁻M⁺, (meth)acryloxy, styryl, styryloxy, vinylbenzyl, or H;
R₇ is H, M⁺, benzyl, CₙH₂ₙ₊₁CH═CHCH₂—, CₙH₂ₙ₊₁CH═C(CH₃)CH₂—, or oxyC₁₋₄ (meth)alkyl, CH₂═CR′—C(═O)—O—(CH₂)ₙ₁—O—C(═O)—O—;
R′ is H or CH₃;
with the proviso that R₁, R₃, R₆ or R₇ have at least one polymerizable double bond;
R₈ is ═NH₂, ═NHCH₂CH₃, ═N(CH₂CH₃)₂, H, ═O, OH, O⁻M⁺, NH₂, C₁-C₄alkyl, —COOH, —CH₂COOH; —COO⁻M⁺, —CH₂COO⁻M⁺,
R₉ is H or CH₃;
R₁₀ is H, —OH, —O⁻M⁺, C₁-C₄alkyl, —COOH, —CH₂COOH, —COO⁻M⁺, or —CH₂COO⁻M⁺;
or R₁₀ is 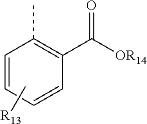
where the dotted bond joins R₁₀ to the remainder of Structure VI;
R₁₂ is =NH₂⁺X⁻, ═NH⁺CH₂CH₃X⁻, ═N⁺(CH₂CH₃)₂X⁻, or =0;
R₁₃ is —NH₂, —COOH, —COO⁻M⁺, or H;
R₁₄ is H or M⁺;
n is 0-10;
m is 2 when n is 0; and m is 1 when n is 0-10;
M⁺ is a cationic counterion; and
n1 is 2-4.

7. The fluorescent monomer according to claim 6, which has the Structure VC and is substantially free of the Structure VIC, wherein Structure VC is: 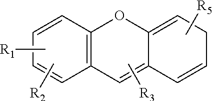
and Structure VIC is:

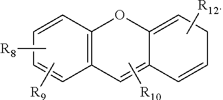

8. The fluorescent monomer according to claim 7, which has the Structure VC-1 and is substantially free of the Structure VIC-1, wherein Structure VC-1 is: 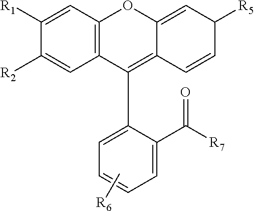
and Structure VIC-1 is:

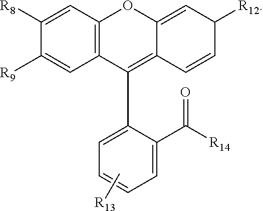

9. The fluorescent monomer according to claim 7, which has the Structure VC-2 and is substantially free of the Structure VIC-2, wherein VC-2 is: 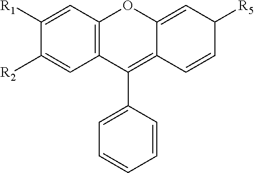
and Structure VCI-2 is:

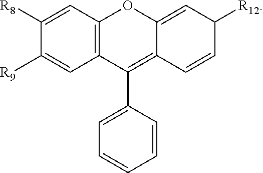

10. The fluorescent monomer according to claim 6, which has the Structure VII and is substantially free of Structure VIII, wherein Structure VII is: 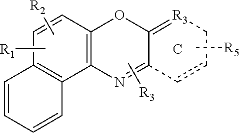
and Structure VIII is:

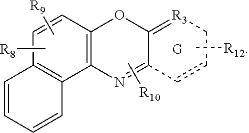

11. The fluorescent monomer according to claim 10, which has the Structure VII-1 and is substantially free of the Structure VIII-1, wherein Structure VII-1 is: 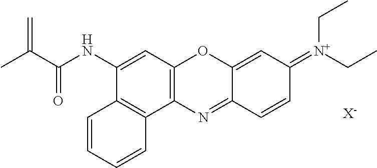
and Structure VIII-1 is:

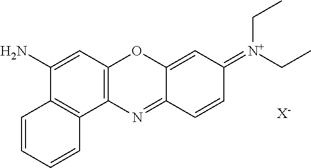

12. A polymer prepared by polymerizing the fluorescent monomer of claim 1 with other monomers.

13. The polymer according to claim 12, wherein the other monomers are selected from the group consisting of cationic, anionic, and/or nonionic monomers.

14. The polymer according to claim 13, wherein the other monomers comprise anionic monomers.

15. The polymer according to claim 14, wherein the anionic monomers comprise carboxylic acid monomers, or salts or anhydrides thereof.

16. The polymer according to claim 12, wherein the fluorescent monomer is incorporated into the polymer to an extent equal to or greater than 90%.

17. A process of preparing a water treatment polymer, the process comprising the following steps:
(a) polymerizing a polymerization mixture comprising:
(i) at least one water-soluble carboxylic acid monomer, or salt or anhydride thereof, present in an amount of 10-99.999 mol % based on 100 mol % of the polymer; and
(ii) at least one fluorescent monomer according to claim 1 to yield said water treatment polymer; and
(b) ensuring the fluorescent monomer is incorporated into the water treatment polymer to an extent equal to or greater than 90%.

18. A method of controlling scale in a water system, the method comprising the steps of:
(a) dosing the water system with a water treatment polymer containing anionic monomers according to claim 14; and
(b) monitoring the fluorescent signal emitted from said water system.

19. A method of suppressing corrosion in a water system, the method comprising the following steps of:
(a) dosing the water system with a water treatment polymer containing anionic monomers according to claim 14; and
(b) monitoring the fluorescent signal emitted from said water system.

20. A method for coagulation or flocculation in a water treatment system, the method comprising the steps of:
(a) dosing the water treatment system with the water treatment polymer according to claim 14; and
(b) monitoring the fluorescent signal emitted from the water treatment system.

21. A method for determining whether a given location has been cleaned comprising the steps of:
(a) applying the polymer according to claim 14 to the location;
(b) cleaning the location at least once; and
(c) attempting to detect the presence of fluorescent monomer incorporated into the polymer remaining at the location after said cleaning, which, presence, if detected, indicates that additional cleaning is needed.
